(19) United States
(12) Patent Application Publication
(10) Pub. No.: US 2025/0114482 A1
(43) Pub. Date: Apr. 10, 2025

(54) THERAPEUTIC APPLICATIONS OF CPF1-BASED GENOME EDITING

(71) Applicant: Duke University, Durham, NC (US)

(72) Inventors: Charles A. Gersbach, Chapel Hill, NC (US); Sarina Madhavan, Katy, TX (US); Christopher Nelson, Durham, NC (US)

(21) Appl. No.: 18/957,197

(22) Filed: Nov. 22, 2024

Related U.S. Application Data
(63) Continuation of application No. 16/318,745, filed on Jan. 18, 2019, now Pat. No. 12,214,056.
(60) Provisional application No. 62/363,888, filed on Jul. 19, 2016.

(51) Int. Cl. C07K 14/47; A61K 31/7105; A61K 38/46; C12N 9/22; C12N 15/11

(52) U.S. Cl. CPC A61K 48/0066; A61K 31/7105; A61K 38/465; A61K 48/0008; A61K 48/0058; A61K 48/0075; A61K 48/0091; A61P 21/00; C07K 14/4708; C12N 9/22; C12N 15/111; C12N 15/113; C12N 15/907; C12N 2310/20; C12N 2320/33; C12N 2750/14143; Y02A 50/30

(57) ABSTRACT

Disclosed herein are therapeutic applications of CRISPR/Cpf1-based genome editing.

### CROSS REFERENCE TO RELATED APPLICATIONS

This application is a continuation of U.S. patent application Ser. No. 16/318,745, filed Jan. 18, 2019, which is the U.S. national stage entry, under 35 U.S.C. § 371, of International Application Number PCT/US2017/042921, filed Jul. 19, 2017, which is the claims priority to U.S. Provisional Application No. 62/363,888, filed Jul. 19, 2016, the entire contents of each of which are hereby incorporated by reference.

### STATEMENT OF GOVERNMENT INTEREST

This invention was made with government support under Federal Grant Nos. AR069085 and MD140071 awarded by the NIH and Army/MRMC, respectively. The U.S. Government has certain rights to this invention.

### SEQUENCE LISTING

The instant application includes a Sequence Listing which has been submitted electronically in ASCII format and is hereby incorporated by reference in its entirety. Said ASCII copy, created on Jul. 19, 2017, is named 028193-9250-WO00 Sequence Listing.txt and is 46,056 bytes in size.

### TECHNICAL FIELD

The present disclosure relates to the field of gene expression alteration, genome engineering and genomic alteration of genes using Clustered Regularly Interspaced Short Palindromic Repeats from Prevotella and Francisella 1 (CRISPR/Cpf1) based systems and viral delivery systems.

### BACKGROUND

RNA-guided nucleases have been adapted for genome modification in human cells including CRISPR/Cpf1 systems derived from Streptococcus pyogenes and Staphylococcus aureus. Numerous microorganisms have been shown to have DNA-editing or RNA-editing systems. Cas9 derived from S. pyogenes and S. aureus make blunt-ended double-stranded breaks (DSBs) through genomic DNA which are repaired by non-homologous end-joining (NHEJ) leaving small insertions and deletions (indels) at the repaired site or through homology directed repair in the presence of a template. These indels can be used to knockout a gene, remove a splice acceptor, or dissect genetic regulatory elements.

Hereditary genetic diseases have devastating effects on children in the United States. These diseases currently have no cure and can only be managed by attempts to alleviate the symptoms. For decades, the field of gene therapy has promised a cure to these diseases. However technical hurdles regarding the safe and efficient delivery of therapeutic genes to cells and patients have limited this approach. Duchenne muscular dystrophy (DMD) is a fatal genetic disease, clinically characterized by muscle wasting, loss of ambulation, and death typically in the third decade of life due to the loss of functional dystrophin. DMD is the result of inherited or spontaneous mutations in the dystrophin gene. Most mutations causing DMD are a result of deletions of exon(s), pushing the translational reading frame out of frame.

Dystrophin is a key component of a protein complex that is responsible for regulating muscle cell integrity and function. DMD patients typically lose the ability to physically support themselves during childhood, become progressively weaker during the teenage years, and die in their twenties. Current experimental gene therapy strategies for DMD require repeated administration of transient gene delivery vehicles or rely on permanent integration of foreign genetic material into the genomic DNA. Both of these methods have serious safety concerns. Furthermore, these strategies have been limited by an inability to deliver the large and complex dystrophin gene sequence. There remains a need for more precise and efficient gene editing tools for correcting and treating patients with mutations in the dystrophin gene.

### SUMMARY

The present invention is directed to a Cpf1 guide RNA (gRNA) that targets a dystrophin gene and comprises a polynucleotide sequence corresponding to at least one of SEQ ID NOs: 36-64, 71-119, or a complement thereof.

The present invention is directed to a DNA targeting composition comprising a Cpf1 endonuclease and at least one Cpf1 gRNA described above.

The present invention is directed to a DNA targeting composition comprising a first Cpf1 gRNA and a second Cpf1 gRNA, the first Cpf1 gRNA and the second Cpf1 gRNA each comprising a polynucleotide sequence corresponding to at least one of SEQ ID NOs: 36-64, 71-119, or a complement thereof, wherein the first Cpf1 gRNA and the second Cpf1 gRNA comprise different polynucleotide sequences, and wherein the first Cpf1 gRNA and the second Cpf1 gRNA target a dystrophin gene.

The present invention is directed to an isolated polynucleotide comprising the Cpf1 gRNA described above or a polynucleotide sequence encoding the DNA targeting composition described above.

The present invention is directed to a vector comprising the Cpf1 gRNA described above, a polynucleotide sequence encoding the DNA targeting composition described above, or the isolated polynucleotide described above.

The present invention is directed to a vector encoding: (a) a first Cpf1 guide RNA (gRNA), (b) a second Cpf1 gRNA, and (c) at least one Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), wherein the first Cpf1 gRNA and the second Cpf1 gRNA comprises a polynucleotide sequence corresponding to at least one of SEQ ID NOs: 36-64, 71-119, or a complement thereof, and wherein the first Cpf1 gRNA and the second Cpf1 gRNA comprise different polynucleotide sequences.

The present invention is directed to a cell comprising the Cpf1 gRNA described above, a polynucleotide sequence encoding the DNA targeting composition described above, the isolated polynucleotide described above, or the vector described above.

The present invention is directed to a kit comprising the Cpf1 gRNA described above, a polynucleotide sequence encoding the DNA targeting composition described above, the isolated polynucleotide described above, the vector described above, or the cell described above.

The present invention is directed to a composition for deleting a segment of a dystrophin gene comprising exon 51, the composition comprising: (a) a first vector comprising a polynucleotide sequence encoding a first Cpf1 guide RNA (gRNA) and a polynucleotide sequence encoding a first Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), and (b) a second vector comprising a polynucleotide sequence encoding a second Cpf1 gRNA and a polynucleotide sequence encoding a second Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), wherein the first Cpf1 gRNA and the second Cpf1 gRNA comprise a polynucleotide sequence corresponding to at least one of SEQ ID NOs: 36-64, 71-119, or a complement thereof, wherein the first Cpf1 gRNA and the second Cpf1 gRNA comprise different polynucleotide sequences, and wherein the first vector and second vector are configured to form a first and a second double strand break in a first intron and a second intron flanking exon 51 of the human DMD gene, respectively, thereby deleting a segment of the dystrophin gene comprising exon 51.

The present invention is directed to a cell comprising the composition described above.

The present invention is directed to a modified adeno-associated viral vector for genome editing a mutant dystrophin gene in a subject comprising a first polynucleotide sequence encoding the Cpf1 gRNA described above, and a second polynucleotide sequence encoding a Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123).

The present invention is directed to a method of correcting a mutant dystrophin gene in a cell, the method comprising administering to a cell the Cpf1 gRNA described above, a polynucleotide sequence encoding the DNA targeting composition described above, the isolated polynucleotide described above, the vector described above, the composition described above, or the modified adeno-associated viral vector described above.

The present invention is directed to a method of genome editing a mutant dystrophin gene in a subject, the method comprising administering to the subject a genome editing composition comprising the Cpf1 gRNA described above, a polynucleotide sequence encoding the DNA targeting composition described above, the isolated polynucleotide described above, the vector described above, the composition described above, or the modified adeno-associated viral vector described above.

The present invention is directed to a method of treating a subject in need thereof having a mutant dystrophin gene, the method comprising administering to the subject the Cpf1 gRNA described above, a polynucleotide sequence encoding the DNA targeting composition described above, the isolated polynucleotide described above, the vector described above, the composition described above, or the modified adeno-associated viral vector described above.

The present invention is directed to a method of correcting a mutant dystrophin gene in a cell, comprising administering to the cell: (a) a first vector comprising a polynucleotide sequence encoding a first Cpf1 guide RNA (gRNA) and a polynucleotide sequence encoding a first Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), and (b) a second vector comprising a polynucleotide sequence encoding a second Cpf1 gRNA and a polynucleotide sequence encoding a second Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), wherein the first Cpf1 gRNA and the second Cpf1 gRNA comprises a polynucleotide sequence corresponding to at least one of SEQ ID NOs: 36-64, 71-119, or a complement thereof, and the vector is configured to form a first and a second double strand break in a first and a second intron flanking exon 51 of the human dystrophin gene, respectively, thereby deleting a segment of the dystrophin gene comprising exon 51 and correcting the mutant dystrophin gene in a cell.

The present invention is directed to a method of treating a subject in need thereof having a mutant dystrophin gene, the method comprising administering to the subject: (a) a first vector comprising a polynucleotide sequence encoding a first Cpf1 guide RNA (gRNA) and a polynucleotide sequence encoding a first Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), and (b) a second vector comprising a polynucleotide sequence encoding a second Cpf1 gRNA and a polynucleotide sequence encoding a second Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), wherein the first Cpf1 gRNA and the second Cpf1 gRNA comprises a polynucleotide sequence corresponding to at least one of SEQ ID NOs: 36-64, 71-119, or a complement thereof, and the first vector and the second vector are configured to form a first and a second double strand break in a first and a second intron flanking exon 51 of the human dystrophin gene, respectively, thereby deleting a segment of the dystrophin gene comprising exon 51 and treating the subject.

The present invention is directed to a Cpf1 guide RNA (gRNA) that targets an enhancer of the B-cell lymphoma/leukemia 11A (BCL11a) gene and comprises a polynucleotide sequence corresponding to at least one of SEQ ID NOs: 65-70, or a complement thereof.

The present invention is directed to a method of disrupting an enhancer of a B-cell lymphoma/leukemia 11A gene in a cell, the method comprising administering to the cell at least one Cpf1 gRNA described above and a Cpf1 endonuclease.

### BRIEF DESCRIPTION OF THE DRAWINGS

FIG. 1 is a schematic drawing showing the use of Cpf1 in three methods of treatment for genetic diseases, such as DMD and SCA/beta thalassemia, in accordance with some embodiments of the present disclosure.

FIG. 2 shows blots showing exon 44, 46 and 51 are targeted gRNAs with detectable activity in accordance with several embodiments of the present disclosure.

FIG. 3 shows a blot showing 42 guide RNA pairs are screened targeting exon 51 deletion in accordance with one embodiment of the present disclosure.

FIG. 4 shows SaCas9 and LbCpf1 are expressed in patient derived myoblasts.

FIG. 5 shows genomic deletions generated by SaCas9 or LbCpf1 in patient myoblasts.

FIG. 6 shows SaCas9 or LbCpf1 targeting exon 51 remove the exon from the transcript.

FIG. 7 illustrates a panel of Cpf1 crRNAs showing surveyor nuclease activity throughout the exon.

### DETAILED DESCRIPTION

The present disclosure provides, in part, therapeutic applications of CRISPR/Cpf1-based genome editing for the treatment of diseases. Cpf1, a type V CRISPR-Cas effector endonuclease, is involved in the adaptive immunity of prokaryotes, including Acidaminococcus and Lachnospiraceae among others, and exhibits gene-editing activity in human cells through a single RNA-guided approach. The present disclosure provides methods in which the CRISPR/Cpf1-based system can be used in the treatment of genetic diseases, such as Duchenne muscular dystrophy (DMD), sickle cell anemia (SCA) and β-thalassemia.

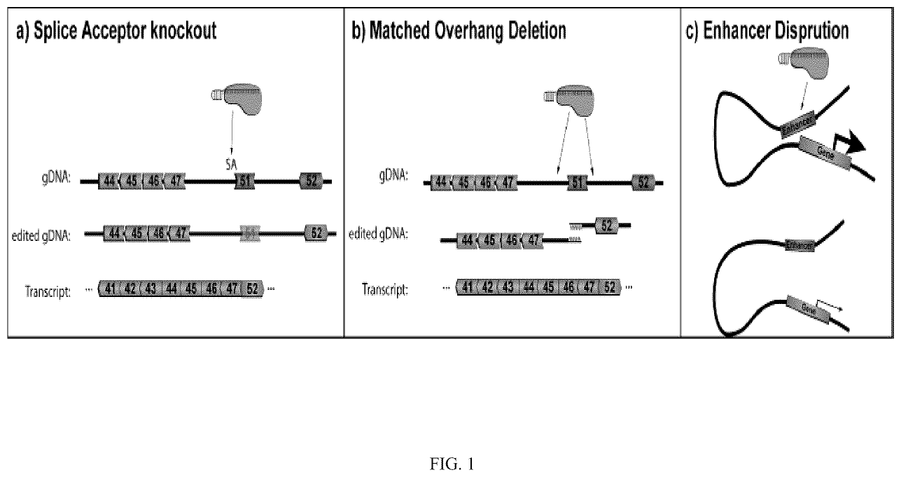

According to one aspect of the present disclosure, the first method comprises a splice acceptor knockout. Cpf1 produces a larger indel footprint making efficient disruption of splice acceptors and removal of target exons from the transcript (see FIG. 1A). As shown in FIG. 1A, Cpf1 generates a 5-base-pair staggered double-stranded break through the DNA, which may be repaired through non-homologous end joining (NHEJ) and produce a larger insertion or deletion (indel) foot print then S. pyogenes or S. aureus Cas9. This will allow for more powerful disruption of splice acceptors and removal of targeted exons as the repair may leave a larger indel footprint making knockout of genetic elements, such as splice acceptors and enhancers, more efficient. Cpf1 also has a distinct protospacer-adjacent motif (PAM) sequence that increases the diversity of genomic regions that can be targeted. Cpf1 recognizes TTTN whereas S. pyogenes Cas9 recognizes NGG and S. aureus Cas9 recognizes NNGRRT. In addition, Cpf1 does not need a tracrRNA, therefore, only crRNA is required, thus also using a small guide RNA.

Another aspect of the present disclosure provides a method comprising a matched overhang deletion. Cpf1 can encourage genetic deletions through matching overhangs to remove genetic elements (see FIG. 1B). As shown in FIG. 1B, Cpf1 generates a 5-base-pair overhang that can be matched with a second double stranded break. Multiplexed Cpf1 guide RNAs can be provided with matched overhangs to encourage seamless genetic deletions. Previous work with S. aureus Cas9 has shown ˜67% of genetic deletions are seamless with one guide RNA pair. For example, matched overhangs generated by multiplexing Cpf1 around a genetic region of interest (e.g. exon 51 in dystrophin) can encourage seamless deletions. After NHEJ, genetic deletions are made that can restore the reading frame of a mutated gene. By matching the overhangs, very precise ligations could be encouraged.

Yet another aspect of the present disclosure provides a method comprising an enhancer disruption. Cpf1 can produce a larger indel footprint making disruption of enhancers and other genetic regulatory elements more probable (see FIG. 1C). As shown in FIG. 1C, the larger indel footprint generated by Cpf1 could also be harnessed to disrupt enhancers to study enhancer function or as a potential treatment for diseases, such as SCA.

For example, the present disclosure describes the adaption of Cpf1 for the targeted genetic removal of single and multiple exons of the dystrophin gene for the treatment of Duchenne muscular dystrophy (DMD). This is accomplished by targeted mutagenesis of splice acceptors in mutational hotspots for single exon removal or by genetic deletions of single or multiple exons. Through targeted exon removal, the reading frame of dystrophin can be restored leading to improved muscle function and patient phenotype. Genetic enhancers can also be targeted as a therapeutic approach to treating disease, specifically targeting the BCL11a enhancer region or gamma globin promoter as a treatment for sickle cell anemia (SCA) or β-thalassemia. The disclosed Cpf1 gRNAs can be used with the CRISPR/Cpf1-based system to target genetic regions, such as intronic regions surrounding exon 51 of the human dystrophin gene, causing genomic deletions of this region in order to restore expression of functional dystrophin in cells from DMD patients.

Also described herein are genetic constructs, compositions and methods for delivering CRISPR/Cpf1-based gene editing system and multiple gRNAs to target the dystrophin gene. The presently disclosed subject matter also provides for methods for delivering the genetic constructs (e.g., vectors) or compositions comprising thereof to skeletal muscle. The vector can be an AAV, including modified AAV vectors. The presently disclosed subject matter describes a way to deliver active forms of this class of therapeutics to skeletal muscle that is effective, efficient and facilitates successful genome modification, as well as provide a means to rewrite the human genome for therapeutic applications and target model species for basic science applications.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Binding region” as used herein refers to the region within a nuclease target region that is recognized and bound by the nuclease.

“Cardiac muscle” or “heart muscle” as used interchangeably herein means a type of involuntary striated muscle found in the walls and histological foundation of the heart, the myocardium. Cardiac muscle is made of cardiomyocytes or myocardiocytes. Myocardiocytes show striations similar to those on skeletal muscle cells but contain only one, unique nucleus, unlike the multinucleated skeletal cells. In certain embodiments, “cardiac muscle condition” refers to a condition related to the cardiac muscle, such as cardiomyopathy, heart failure, arrhythmia, and inflammatory heart disease.

“Coding sequence” or “encoding nucleic acid” as used herein means the nucleic acids (RNA or DNA molecule) that comprise a polynucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimize.

“Complement” or “complementary” as used herein means a nucleic acid can mean Watson-Crick (e.g., A-T/U and C-G) or Hoogsteen base pairing between polynucleotides or polynucleotide analogs of nucleic acid molecules. “Complementarity” refers to a property shared between two nucleic acid sequences, such that when they are aligned antiparallel to each other, the polynucleotide bases at each position will be complementary.

“Correcting”, “genome editing” and “restoring” as used herein refers to changing a mutant gene that encodes a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Cpf1 endonuclease” or “Cpf1” as used interchangeably herein refers to a single RNA-Guided endonuclease of a Class 2 CRISPR-Cas system that is a smaller and a simpler endonuclease than Cas9. The Cpf1 endonuclease targets and cleaves as a 5-nucleotide staggered cut distal to a 5′T-rich PAM.

“Donor DNA”, “donor template” and “repair template” as used interchangeably herein refers to a double-stranded DNA fragment or molecule that includes at least a portion of the gene of interest. The donor DNA may encode a full-functional protein or a partially-functional protein.

“Duchenne Muscular Dystrophy” or “DMD” as used interchangeably herein refers to a recessive, fatal, X-linked disorder that results in muscle degeneration and eventual death. DMD is a common hereditary monogenic disease and occurs in 1 in 3500 males. DMD is the result of inherited or spontaneous mutations that cause nonsense or frame shift mutations in the dystrophin gene. The majority of dystrophin mutations that cause DMD are deletions of exons that disrupt the reading frame and cause premature translation termination in the dystrophin gene. DMD patients typically lose the ability to physically support themselves during childhood, become progressively weaker during the teenage years, and die in their twenties.

“Dystrophin” as used herein refers to a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb. 79 exons code for the protein which is over 3500 amino acids.

“Exon 51” as used herein refers to the 51^{st} exon of the dystrophin gene. Exon 51 is frequently adjacent to frame-disrupting deletions in DMD patients and has been targeted in clinical trials for oligonucleotide-based exon skipping. A clinical trial for the exon 51 skipping compound eteplirsen recently reported a significant functional benefit across 48 weeks, with an average of 47% dystrophin positive fibers compared to baseline. Mutations in exon 51 are ideally suited for permanent correction by NHEJ-based genome editing.

“Frameshift” or “frameshift mutation” as used interchangeably herein refers to a type of gene mutation wherein the addition or deletion of one or more polynucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Genetic construct” as used herein refers to the DNA or RNA molecules that comprise a polynucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, Becker Muscular Dystrophy (BMD), hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, such as β-thalassemia, xeroderma pigmentosum, Fanconi's anemia, retinitis pigmentosa, ataxia telangiectasia, Bloom's syndrome, retinoblastoma, and Tay-Sachs disease.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cpf1-based gene editing system, then the cellular machinery will repair the break by homologous recombination, which is enhanced several orders of magnitude in the presence of DNA cleavage. When the homologous DNA piece is absent, non-homologous end joining may take place instead.

“Genome editing” as used herein refers to changing a gene. Genome editing may include correcting or restoring a mutant gene. Genome editing may include knocking out a gene, such as a mutant gene or a normal gene. Genome editing may be used to treat disease or enhance muscle repair by changing the gene of interest.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of polynucleotides may also occur, but is much more common when the overhangs are not compatible.

“Normal gene” as used herein refers to a gene that has not undergone a change, such as a loss, gain, or exchange of genetic material. The normal gene undergoes normal gene transmission and gene expression.

“Nuclease mediated NHEJ” as used herein refers to NHEJ that is initiated after a nuclease, such as a Cpf1 endonuclease, cuts double stranded DNA.

“Nucleic acid” or “oligonucleotide” or “polynucleotide” as used herein means at least two polynucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Partially-functional” as used herein describes a protein that is encoded by a mutant gene and has less biological activity than a functional protein but more than a non-functional protein.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter.

“Skeletal muscle” as used herein refers to a type of striated muscle, which is under the control of the somatic nervous system and attached to bones by bundles of collagen fibers known as tendons. Skeletal muscle is made up of individual components known as myocytes, or “muscle cells”, sometimes colloquially called “muscle fibers.” Myocytes are formed from the fusion of developmental myoblasts (a type of embryonic progenitor cell that gives rise to a muscle cell) in a process known as myogenesis. These long, cylindrical, multinucleated cells are also called myofibers.

“Skeletal muscle condition” as used herein refers to a condition related to the skeletal muscle, such as muscular dystrophies, aging, muscle degeneration, wound healing, and muscle weakness or atrophy.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

“Target gene” as used herein refers to any polynucleotide sequence encoding a known or putative gene product. The target gene may be a mutated gene involved in a genetic disease. In certain embodiments, the target gene is a human dystrophin gene or a human B-cell lymphoma/leukemia 11A gene. In certain embodiments, the target gene is a mutant human dystrophin gene.

“Target region” as used herein refers to the region of the target gene to which the CRISPR/Cpf1-based gene editing system is designed to bind and cleave.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Variant” used herein with respect to a nucleic acid means (i) a portion or fragment of a referenced polynucleotide sequence; (ii) the complement of a referenced polynucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid. For example, the vector may encode a Cpf1 endonuclease and at least one Cpf1 gRNA, such as a Cpf1 gRNA comprising a polynucleotide sequence of any one of SEQ ID NOs: 36-119, or complement thereof.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. CRISPR System

A presently disclosed genetic construct (e.g., a vector) encodes a CRISPR/Cpf1-based gene editing system that is specific for a dystrophin gene (e.g., human dystrophin gene). “Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a ‘memory’ of past exposures.

Three classes of CRISPR systems (Types I, II and III effector systems) are known. The Type II effector system carries out targeted DNA double-strand break in four sequential steps, using a single effector enzyme, such as a Cpf1 endonuclease, to cleave dsDNA. Compared to the Type I and Type III effector systems, which require multiple distinct effectors acting as a complex, the Type II effector system may function in alternative contexts such as eukaryotic cells. Cpf1 endonuclease mediates cleavage of target DNA if a correct PAM is also present on the 5′ end of the protospacer.

CRISPR/Cpf1 systems activity has three stages: adaptation, formation of crRNAs, and interference. During adaptation, Cas1 and Cas2 proteins facilitate the adaptation of small fragments of DNA into the CRISPR array. The processing of pre-cr-RNAs occurs during formation of crRNAs to produce mature crRNAs to guide the Cas protein, i.e., the Cpf1 endonuclease. During interference: the Cpf1 is bound to a crRNA to form a binary complex to identify and cleave a target DNA sequence.

In this system, the Cpf1 endonuclease is directed to genomic target sites by a synthetically reconstituted Cpf1 “guide RNA” (“Cpf1 gRNA”). The Cpf1 endonuclease leaves one strand longer than the other, creating ‘sticky’ ends, for example 4-5 nucleotide long sticky ends, unlike Cas9 which generates blunt ends. The Cpf1 endonuclease also cleaves target DNA further away from PAM compared to Cas9.

The target gene (e.g., a dystrophin gene, e.g., human dystrophin gene) can be involved in differentiation of a cell or any other process in which activation of a gene can be desired, or can have a mutation such as a frameshift mutation or a nonsense mutation. If the target gene has a mutation that causes a premature stop codon, an aberrant splice acceptor site or an aberrant splice donor site, the CRISPR/Cpf1-based gene editing system can be designed to recognize and bind a polynucleotide sequence upstream or downstream from the premature stop codon, the aberrant splice acceptor site or the aberrant splice donor site. The CRISPR/Cpf1-based system can also be used to disrupt normal gene splicing by targeting splice acceptors and donors to induce skipping of premature stop codons or restore a disrupted reading frame. The CRISPR/Cpf1-based gene editing system may or may not mediate off-target changes to protein-coding regions of the genome.

Provided herein are CRISPR/Cpf1-based engineered systems for use in genome editing and treating genetic diseases. A unique capability of the CRISPR/Cpf1-based gene editing system is the straightforward ability to simultaneously target multiple distinct genomic loci by co-expressing a single Cpf1 endonuclease with two or more Cpf1 gRNAs. The CRISPR/Cpf1-based engineered systems can be designed to target any gene, including genes involved in a genetic disease, aging, tissue regeneration, or wound healing. The CRISPR/Cpf1-based gene editing systems can include a Cpf1 endonuclease and at least one Cpf1 gRNA. In certain embodiments, the system comprises two Cpf1 gRNAs.

a. Cpf1 Endonuclease

The CRISPR/Cpf1-based gene editing system can include a Cpf1 endonuclease. Cpf1 endonuclease is an endonuclease that cleaves nucleic acid. The Cpf1 endonuclease cleaves in a staggered fashion, creating a 5 nucleotide 5′ overhang 18-23 bases away from the PAM, whereas Cas9 generates blunt ends 3 nucleotide upstream of the PAM site. The Cpf1 endonuclease can be from any bacterial or archaea species, including, but not limited to, Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33 10, Parcubacteria bacterium GW2011 GWC2 44 17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens or Porphyromonas macacae. In certain embodiments, the Cpf1 endonuclease is a Cpf1 endonuclease from Lachnospiracede bacterium ND2006 (“LbCpf1”) or from Acidaminococcus (“AsCpf1”).

In some embodiments, the Cpf1 endonuclease can include a humanized AsCpf1 sequence (SEQ ID NO: 124) as follows:

(SEQ ID NO: 124)
gacggatcgggagatctcccgatcccctatggtgcactctcagtacaatctgctctgatgccgca
tagttaagccagtatctgctccctgcttgtgtgttggaggtcgctgagtagtgcgcgagcaaaat
ttaagctacaacaaggcaaggcttgaccgacaattgcatgaagaatctgcttagggttaggcgtt
ttgcgctgcttcgcgatgtacgggccagatatacgcgttgacattgattattgactagttattaa
tagtaatcaattacggggtcattagttcatagcccatatatggagttccgcgttacataacttac
ggtaaatggcccgcctggctgaccgcccaacgacccccgcccattgacgtcaataatgacgtatg
ttcccatagtaacgccaatagggactttccattgacgtcaatgggtggagtatttacggtaaact
gcccacttggcagtacatcaagtgtatcatatgccaagtacgccccctattgacgtcaatgacgg
taaatggcccgcctggcattatgcccagtacatgaccttatgggactttcctacttggcagtaca
tctacgtattagtcatcgctattaccatggtgatgcggttttggcagtacatcaatgggcgtgga
tagcggtttgactcacggggatttccaagtctccaccccattgacgtcaatgggagtttgttttg
gcaccaaaatcaacgggactttccaaaatgtcgtaacaactccgccccattgacgcaaatgggcg
gtaggcgtgtacggtgggaggtctatataagcagagctctctggctaactagagaacccactgct
tactggcttatcgaaattaatacgactcactatagggagacccaagctggctagcgtttaaactt
aagcttggtaccgccaccATGACACAGTTCGAGGGCTTTACCAACCTGTATCAGGTGAGCAAGAC
ACTGCGGTTTGAGCTGATCCCACAGGGCAAGACCCTGAAGCACATCCAGGAGCAGGGCTTCATCG
AGGAGGACAAGGCCCGCAATGATCACTACAAGGAGCTGAAGCCCATCATCGATCGGATCTACAAG
ACCTATGCCGACCAGTGCCTGCAGCTGGTGCAGCTGGATTGGGAGAACCTGAGCGCCGCCATCGA
CTCCTATAGAAAGGAGAAAACCGAGGAGACAAGGAACGCCCTGATCGAGGAGCAGGCCACATATC
GCAATGCCATCCACGACTACTTCATCGGCCGGACAGACAACCTGACCGATGCCATCAATAAGAGA
CACGCCGAGATCTACAAGGGCCTGTTCAAGGCCGAGCTGTTTAATGGCAAGGTGCTGAAGCAGCT
GGGCACCGTGACCACAACCGAGCACGAGAACGCCCTGCTGCGGAGCTTCGACAAGTTTACAACCT
ACTTCTCCGGCTTTTATGAGAACAGGAAGAACGTGTTCAGCGCCGAGGATATCAGCACAGCCATC
CCACACCGCATCGTGCAGGACAACTTCCCCAAGTTTAAGGAGAATTGTCACATCTTCACACGCCT
GATCACCGCCGTGCCCAGCCTGCGGGAGCACTTTGAGAACGTGAAGAAGGCCATCGGCATCTTCG
TGAGCACCTCCATCGAGGAGGTGTTTTCCTTCCCTTTTTATAACCAGCTGCTGACACAGACCCAG
ATCGACCTGTATAACCAGCTGCTGGGAGGAATCTCTCGGGAGGCAGGCACCGAGAAGATCAAGGG
CCTGAACGAGGTGCTGAATCTGGCCATCCAGAAGAATGATGAGACAGCCCACATCATCGCCTCCC
TGCCACACAGATTCATCCCCCTGTTTAAGCAGATCCTGTCCGATAGGAACACCCTGTCTTTCATC
CTGGAGGAGTTTAAGAGCGACGAGGAAGTGATCCAGTCCTTCTGCAAGTACAAGACACTGCTGAG
AAACGAGAACGTGCTGGAGACAGCCGAGGCCCTGTTTAACGAGCTGAACAGCATCGACCTGACAC
ACATCTTCATCAGCCACAAGAAGCTGGAGACAATCAGCAGCGCCCTGTGCGACCACTGGGATACA
CTGAGGAATGCCCTGTATGAGCGGAGAATCTCCGAGCTGACAGGCAAGATCACCAAGTCTGCCAA
GGAGAAGGTGCAGCGCAGCCTGAAGCACGAGGATATCAACCTGCAGGAGATCATCTCTGCCGCAG
GCAAGGAGCTGAGCGAGGCCTTCAAGCAGAAAACCAGCGAGATCCTGTCCCACGCACACGCCGCC
CTGGATCAGCCACTGCCTACAACCCTGAAGAAGCAGGAGGAGAAGGAGATCCTGAAGTCTCAGCT
GGACAGCCTGCTGGGCCTGTACCACCTGCTGGACTGGTTTGCCGTGGATGAGTCCAACGAGGTGG
ACCCCGAGTTCTCTGCCCGGCTGACCGGCATCAAGCTGGAGATGGAGCCTTCTCTGAGCTTCTAC
AACAAGGCCAGAAATTATGCCACCAAGAAGCCCTACTCCGTGGAGAAGTTCAAGCTGAACTTTCA
GATGCCTACACTGGCCTCTGGCTGGGACGTGAATAAGGAGAAGAACAATGGCGCCATCCTGTTTG
TGAAGAACGGCCTGTACTATCTGGGCATCATGCCAAAGCAGAAGGGCAGGTATAAGGCCCTGAGC
TTCGAGCCCACAGAGAAAACCAGCGAGGGCTTTGATAAGATGTACTATGACTACTTCCCTGATGC
CGCCAAGATGATCCCAAAGTGCAGCACCCAGCTGAAGGCCGTGACAGCCCACTTTCAGACCCACA
CAACCCCCATCCTGCTGTCCAACAATTTCATCGAGCCTCTGGAGATCACAAAGGAGATCTACGAC
CTGAACAATCCTGAGAAGGAGCCAAAGAAGTTTCAGACAGCCTACGCCAAGAAAACCGGCGACCA
GAAGGGCTACAGAGAGGCCCTGTGCAAGTGGATCGACTTCACAAGGGATTTTCTGTCCAAGTATA
CCAAGACAACCTCTATCGATCTGTCTAGCCTGCGGCCATCCTCTCAGTATAAGGACCTGGGCGAG
TACTATGCCGAGCTGAATCCCCTGCTGTACCACATCAGCTTCCAGAGAATCGCCGAGAAGGAGAT
CATGGATGCCGTGGAGACAGGCAAGCTGTACCTGTTCCAGATCTATAACAAGGACTTTGCCAAGG
GCCACCACGGCAAGCCTAATCTGCACACACTGTATTGGACCGGCCTGTTTTCTCCAGAGAACCTG
GCCAAGACAAGCATCAAGCTGAATGGCCAGGCCGAGCTGTTCTACCGCCCTAAGTCCAGGATGAA
GAGGATGGCACACCGGCTGGGAGAGAAGATGCTGAACAAGAAGCTGAAGGATCAGAAAACCCCAA
TCCCCGACACCCTGTACCAGGAGCTGTACGACTATGTGAATCACAGACTGTCCCACGACCTGTCT
GATGAGGCCAGGGCCCTGCTGCCCAACGTGATCACCAAGGAGGTGTCTCACGAGATCATCAAGGA
TAGGCGCTTTACCAGCGACAAGTTCTTTTTCCACGTGCCTATCACACTGAACTATCAGGCCGCCA
ATTCCCCATCTAAGTTCAACCAGAGGGTGAATGCCTACCTGAAGGAGCACCCCGAGACACCTATC
ATCGGCATCGATCGGGGCGAGAGAAACCTGATCTATATCACAGTGATCGACTCCACCGGCAAGAT
CCTGGAGCAGCGGAGCCTGAACACCATCCAGCAGTTTGATTACCAGAAGAAGCTGGACAACAGGG
AGAAGGAGAGGGTGGCAGCAAGGCAGGCCTGGTCTGTGGTGGGCACAATCAAGGATCTGAAGCAG
GGCTATCTGAGCCAGGTCATCCACGAGATCGTGGACCTGATGATCCACTACCAGGCCGTGGTGGT
GCTGGAGAACCTGAATTTCGGCTTTAAGAGCAAGAGGACCGGCATCGCCGAGAAGGCCGTGTACC
AGCAGTTCGAGAAGATGCTGATCGATAAGCTGAATTGCCTGGTGCTGAAGGACTATCCAGCAGAG
AAAGTGGGAGGCGTGCTGAACCCATACCAGCTGACAGACCAGTTCACCTCCTTTGCCAAGATGGG
CACCCAGTCTGGCTTCCTGTTTTACGTGCCTGCCCCATATACATCTAAGATCGATCCCCTGACCG
GCTTCGTGGACCCCTTCGTGTGGAAAACCATCAAGAATCACGAGAGCCGCAAGCACTTCCTGGAG
GGCTTCGACTTTCTGCACTACGACGTGAAAACCGGCGACTTCATCCTGCACTTTAAGATGAACAG
AAATCTGTCCTTCCAGAGGGGCCTGCCCGGCTTTATGCCTGCATGGGATATCGTGTTCGAGAAGA
ACGAGACACAGTTTGACGCCAAGGGCACCCCTTTCATCGCCGGCAAGAGAATCGTGCCAGTGATC
GAGAATCACAGATTCACCGGCAGATACCGGGACCTGTATCCTGCCAACGAGCTGATCGCCCTGCT
GGAGGAGAAGGGCATCGTGTTCAGGGATGGCTCCAACATCCTGCCAAAGCTGCTGGAGAATGACG
ATTCTCACGCCATCGACACCATGGTGGCCCTGATCCGCAGCGTGCTGCAGATGCGGAACTCCAAT
GCCGCCACAGGCGAGGACTATATCAACAGCCCCGTGCGCGATCTGAATGGCGTGTGCTTCGACTC
CCGGTTTCAGAACCCAGAGTGGCCCATGGACGCCGATGCCAATGGCGCCTACCACATCGCCCTGA
AGGGCCAGCTGCTGCTGAATCACCTGAAGGAGAGCAAGGATCTGAAGCTGCAGAACGGCATCTCC
AATCAGGACTGGCTGGCCTACATCCAGGAGCTGCGCAACAAAAGGCCGGCGGCCACGAAAAAGGC
CGGCCAGGCAAAAAAGAAAAAGGGATCCTACCCATACGATGTTCCAGATTACGCTTATCCCTACG
ACGTGCCTGATTATGCATACCCATATGATGTCCCCGACTATGCCTAAGaattctgcagatatcca
gcacagtggcggccgctcgagtctagagggcccgtttaaacccgctgatcagcctcgactgtgcc
ttctagttgccagccatctgttgtttgcccctcccccgtgccttccttgaccctggaaggtgcca
ctcccactgtcctttcctaataaaatgaggaaattgcatcgcattgtctgagtaggtgtcattct
attctggggggtggggggggcaggacagcaagggggaggattgggaagacaatagcaggcatgct
ggggatgcggtgggctctatggcttctgaggcggaaagaaccagctggggctctagggggtatcc
ccacgcgccctgtagcggcgcattaagcgcggcgggtgtggtggttacgcgcagcgtgaccgcta
cacttgccagcgccctagcgcccgctcctttcgctttcttcccttcctttctcgccacgttcgcc
ggctttccccgtcaagctctaaatcgggggctccctttagggttccgatttagtgctttacggca
cctcgaccccaaaaaacttgattagggtgatggttcacgtagtgggccatcgccctgatagacgg
tttttcgccctttgacgttggagtccacgttctttaatagtggactcttgttccaaactggaaca
acactcaaccctatctcggtctattcttttgatttataagggattttgccgatttcggcctattg
gttaaaaaatgagctgatttaacaaaaatttaacgcgaattaattctgtggaatgtgtgtcagtt
agggtgtggaaagtccccaggctccccagcaggcagaagtatgcaaagcatgcatctcaattagt
cagcaaccaggtgtggaaagtccccaggctccccagcaggcagaagtatgcaaagcatgcatctc
aattagtcagcaaccatagtcccgcccctaactccgcccatcccgcccctaactccgcccagttc
cgcccattctccgccccatggctgactaattttttttatttatgcagaggccgaggccgcctctg
cctctgagctattccagaagtagtgaggaggcttttttggaggcctaggcttttgcaaaaagctc
ccgggagcttgtatatccattttcggatctgatcaagagacaggatgaggatcgtttcgcatgat
tgaacaagatggattgcacgcaggttctccggccgcttgggtggagaggctattcggctatgact
gggcacaacagacaatcggctgctctgatgccgccgtgttccggctgtcagcgcaggggcgcccg
gttctttttgtcaagaccgacctgtccggtgccctgaatgaactgcaggacgaggcagcgcggct
atcgtggctggccacgacgggcgttccttgcgcagctgtgctcgacgttgtcactgaagcgggaa
gggactggctgctattgggcgaagtgccggggcaggatctcctgtcatctcaccttgctcctgcc
gagaaagtatccatcatggctgatgcaatgggggctgcatacgcttgatccggctacctgcccat
tcgaccaccaagcgaaacatcgcatcgagcgagcacgtactcggatggaagccggtcttgtcgat
caggatgatctggacgaagagcatcaggggctcgcgccagccgaactgttcgccaggctcaaggc
gcgcatgcccgacggcgaggatctcgtcgtgacccatggcgatgcctgcttgccgaatatcatgg
tggaaaatggccgcttttctggattcatcgactgtggccggctgggtgtggcggaccgctatcag
gacatagcgttggctacccgtgatattgctgaagagcttggcggcgaatgggctgaccgcttcct
cgtgctttacggtatcgccgctcccgattcgcagcgcatcgccttctatcgccttcttgacgagt
tcttctgagcgggactctggggttcgaaatgaccgaccaagcgacgcccaacctgccatcacgag
atttcgattccaccgccgccttctatgaaaggttgggcttcggaatcgttttccgggacgccggc
tggatgatcctccagcgcggggatctcatgctggagttcttcgcccaccccaacttgtttattgc
agcttataatggttacaaataaagcaatagcatcacaaatttcacaaataaagcatttttttcac
tgcattctagttgtggtttgtccaaactcatcaatgtatcttatcatgtctgtataccgtcgacc
tctagctagagcttggcgtaatcatggtcatagctgtttcctgtgtgaaattgttatccgctcac
aattccacacaacatacgagccggaagcataaagtgtaaagcctggggtgcctaatgagtgagct
aactcacattaattgcgttgcgctcactgcccgctttccagtcgggaaacctgtcgtgccagctg
cattaatgaatcggccaacgcgcggggagaggcggtttgcgtattgggcgctcttccgcttcctc
gctcactgactcgctgcgctcggtcgttcggctgcggcgagcggtatcagctcactcaaaggcgg
taatacggttatccacagaatcaggggataacgcaggaaagaacatgtgagcaaaaggccagcaa
aaggccaggaaccgtaaaaaggccgcgttgctggcgtttttccataggctccgcccccctgacga
gcatcacaaaaatcgacgctcaagtcagaggtggcgaaacccgacaggactataaagataccagg
cgtttccccctggaagctccctcgtgcgctctcctgttccgaccctgccgcttaccggatacctg
tccgcctttctcccttcgggaagcgtggcgctttctcatagctcacgctgtaggtatctcagttc
ggtgtaggtcgttcgctccaagctgggctgtgtgcacgaaccccccgttcagcccgaccgctgcg
ccttatccggtaactatcgtcttgagtccaacccggtaagacacgacttatcgccactggcagca
gccactggtaacaggattagcagagcgaggtatgtaggcggtgctacagagttcttgaagtggtg
gcctaactacggctacactagaagaacagtatttggtatctgcgctctgctgaagccagttacct
tcggaaaaagagttggtagctcttgatccggcaaacaaaccaccgctggtagcggtttttttgtt
tgcaagcagcagattacgcgcagaaaaaaaggatctcaagaagatcctttgatcttttctacggg
gtctgacgctcagtggaacgaaaactcacgttaagggattttggtcatgagattatcaaaaagga
tcttcacctagatccttttaaattaaaaatgaagttttaaatcaatctaaagtatatatgagtaa
acttggtctgacagttaccaatgcttaatcagtgaggcacctatctcagcgatctgtctatttcg
ttcatccatagttgcctgactccccgtcgtgtagataactacgatacgggagggcttaccatctg
gccccagtgctgcaatgataccgcgagacccacgctcaccggctccagatttatcagcaataaac
cagccagccggaagggccgagcgcagaagtggtcctgcaactttatccgcctccatccagtctat
taattgttgccgggaagctagagtaagtagttcgccagttaatagtttgcgcaacgttgttgcca
ttgctacaggcatcgtggtgtcacgctcgtcgtttggtatggcttcattcagctccggttcccaa
cgatcaaggcgagttacatgatcccccatgttgtgcaaaaaagcggttagctccttcggtcctcc
gatcgttgtcagaagtaagttggccgcagtgttatcactcatggttatggcagcactgcataatt
ctcttactgtcatgccatccgtaagatgcttttctgtgactggtgagtactcaaccaagtcattc
tgagaatagtgtatgcggcgaccgagttgctcttgcccggcgtcaatacgggataataccgcgcc
acatagcagaactttaaaagtgctcatcattggaaaacgttcttcggggcgaaaactctcaagga
tcttaccgctgttgagatccagttcgatgtaacccactcgtgcacccaactgatcttcagcatct
tttactttcaccagcgtttctgggtgagcaaaaacaggaaggcaaaatgccgcaaaaaagggaat
aagggcgacacggaaatgttgaatactcatactcttcctttttcaatattattgaagcatttatc
agggttattgtctcatgagcggatacatatttgaatgtatttagaaaaataaacaaataggggtt
ccgcgcacatttccccgaaaagtgccacctgacgtc.

In some embodiments, the Cpf1 endonuclease can include a humanized LbCpf1 sequence (SEQ ID NO: 125) as follows:

(SEQ ID NO: 125)
gacggatcgggagatctcccgatcccctatggtgcactctcagtacaatctgctctgatgccgcatagttaagccagtatctg
ctccctgcttgtgtgttggaggtcgctgagtagtgcgcgagcaaaatttaagctacaacaaggcaaggcttgaccgacaattgcatgaagaa
tctgcttagggttaggcgttttgcgctgcttcgcgatgtacgggccagatatacgcgttgacattgattattgactagttattaatagtaatcaa
ttacggggtcattagttcatagcccatatatggagttccgcgttacataacttacggtaaatggcccgcctggctgaccgcccaacgacccccg
cccattgacgtcaataatgacgtatgttcccatagtaacgccaatagggactttccattgacgtcaatgggtggagtatttacggtaaactgcc
cacttggcagtacatcaagtgtatcatatgccaagtacgccccctattgacgtcaatgacggtaaatggcccgcctggcattatgcccagtac
atgaccttatgggactttcctacttggcagtacatctacgtattagtcatcgctattaccatggtgatgcggttttggcagtacatcaatgggcg
tggatagcggtttgactcacggggatttccaagtctccaccccattgacgtcaatgggagtttgttttggcaccaaaatcaacgggactttccaa
aatgtcgtaacaactccgccccattgacgcaaatgggcggtaggcgtgtacggtgggaggtctatataagcagagctctctggctaactag
agaacccactgcttactggcttatcgaaattaatacgactcactatagggagacccaagctggctagcgtttaaacttaagcttggtaccgcca
ccATGAGCAAGCTGGAGAAGTTTACAAACTGCTACTCCCTGTCTAAGACCCTGAGGT
TCAAGGCCATCCCTGTGGGCAAGACCCAGGAGAACATCGACAATAAGCGGCTGCTG
GTGGAGGACGAGAAGAGAGCCGAGGATTATAAGGGCGTGAAGAAGCTGCTGGATC
GCTACTATCTGTCTTTTATCAACGACGTGCTGCACAGCATCAAGCTGAAGAATCTGA
ACAATTACATCAGCCTGTTCCGGAAGAAAACCAGAACCGAGAAGGAGAATAAGGA
GCTGGAGAACCTGGAGATCAATCTGCGGAAGGAGATCGCCAAGGCCTTCAAGGGCA
ACGAGGGCTACAAGTCCCTGTTTAAGAAGGATATCATCGAGACAATCCTGCCAGAG
TTCCTGGACGATAAGGACGAGATCGCCCTGGTGAACAGCTTCAATGGCTTTACCACA
GCCTTCACCGGCTTCTTTGATAACAGAGAGAATATGTTTTCCGAGGAGGCCAAGAGC
ACATCCATCGCCTTCAGGTGTATCAACGAGAATCTGACCCGCTACATCTCTAATATG
GACATCTTCGAGAAGGTGGACGCCATCTTTGATAAGCACGAGGTGCAGGAGATCAA
GGAGAAGATCCTGAACAGCGACTATGATGTGGAGGATTTCTTTGAGGGCGAGTTCTT
TAACTTTGTGCTGACACAGGAGGGCATCGACGTGTATAACGCCATCATCGGCGGCTT
CGTGACCGAGAGCGGCGAGAAGATCAAGGGCCTGAACGAGTACATCAACCTGTATA
ATCAGAAAACCAAGCAGAAGCTGCCTAAGTTTAAGCCACTGTATAAGCAGGTGCTG
AGCGATCGGGAGTCTCTGAGCTTCTACGGCGAGGGCTATACATCCGATGAGGAGGT
GCTGGAGGTGTTTAGAAACACCCTGAACAAGAACAGCGAGATCTTCAGCTCCATCA
AGAAGCTGGAGAAGCTGTTCAAGAATTTTGACGAGTACTCTAGCGCCGGCATCTTTG
TGAAGAACGGCCCCGCCATCAGCACAATCTCCAAGGATATCTTCGGCGAGTGGAAC
GTGATCCGGGACAAGTGGAATGCCGAGTATGACGATATCCACCTGAAGAAGAAGGC
CGTGGTGACCGAGAAGTACGAGGACGATCGGAGAAAGTCCTTCAAGAAGATCGGCT
CCTTTTCTCTGGAGCAGCTGCAGGAGTACGCCGACGCCGATCTGTCTGTGGTGGAGA
AGCTGAAGGAGATCATCATCCAGAAGGTGGATGAGATCTACAAGGTGTATGGCTCC
TCTGAGAAGCTGTTCGACGCCGATTTTGTGCTGGAGAAGAGCCTGAAGAAGAACGA
CGCCGTGGTGGCCATCATGAAGGACCTGCTGGATTCTGTGAAGAGCTTCGAGAATTA
CATCAAGGCCTTCTTTGGCGAGGGCAAGGAGACAAACAGGGACGAGTCCTTCTATG
GCGATTTTGTGCTGGCCTACGACATCCTGCTGAAGGTGGACCACATCTACGATGCCA
TCCGCAATTATGTGACCCAGAAGCCCTACTCTAAGGATAAGTTCAAGCTGTATTTTC
AGAACCCTCAGTTCATGGGCGGCTGGGACAAGGATAAGGAGACAGACTATCGGGCC
ACCATCCTGAGATACGGCTCCAAGTACTATCTGGCCATCATGGATAAGAAGTACGCC
AAGTGCCTGCAGAAGATCGACAAGGACGATGTGAACGGCAATTACGAGAAGATCAA
CTATAAGCTGCTGCCCGGCCCTAATAAGATGCTGCCAAAGGTGTTCTTTTCTAAGAA
GTGGATGGCCTACTATAACCCCAGCGAGGACATCCAGAAGATCTACAAGAATGGCA
CATTCAAGAAGGGCGATATGTTTAACCTGAATGACTGTCACAAGCTGATCGACTTCT
TTAAGGATAGCATCTCCCGGTATCCAAAGTGGTCCAATGCCTACGATTTCAACTTTT
CTGAGACAGAGAAGTATAAGGACATCGCCGGCTTTTACAGAGAGGTGGAGGAGCAG
GGCTATAAGGTGAGCTTCGAGTCTGCCAGCAAGAAGGAGGTGGATAAGCTGGTGGA
GGAGGGCAAGCTGTATATGTTCCAGATCTATAACAAGGACTTTTCCGATAAGTCTCA
CGGCACACCCAATCTGCACACCATGTACTTCAAGCTGCTGTTTGACGAGAACAATCA
CGGACAGATCAGGCTGAGCGGAGGAGCAGAGCTGTTCATGAGGCGCGCCTCCCTGA
AGAAGGAGGAGCTGGTGGTGCACCCAGCCAACTCCCCTATCGCCAACAAGAATCCA
GATAATCCCAAGAAAACCACAACCCTGTCCTACGACGTGTATAAGGATAAGAGGTT
TTCTGAGGACCAGTACGAGCTGCACATCCCAATCGCCATCAATAAGTGCCCCAAGA
ACATCTTCAAGATCAATACAGAGGTGCGCGTGCTGCTGAAGCACGACGATAACCCC
TATGTGATCGGCATCGATAGGGGCGAGCGCAATCTGCTGTATATCGTGGTGGTGGAC
GGCAAGGGCAACATCGTGGAGCAGTATTCCCTGAACGAGATCATCAACAACTTCAA
CGGCATCAGGATCAAGACAGATTACCACTCTCTGCTGGACAAGAAGGAGAAGGAGA
GGTTCGAGGCCCGCCAGAACTGGACCTCCATCGAGAATATCAAGGAGCTGAAGGCC
GGCTATATCTCTCAGGTGGTGCACAAGATCTGCGAGCTGGTGGAGAAGTACGATGC
CGTGATCGCCCTGGAGGACCTGAACTCTGGCTTTAAGAATAGCCGCGTGAAGGTGG
AGAAGCAGGTGTATCAGAAGTTCGAGAAGATGCTGATCGATAAGCTGAACTACATG
GTGGACAAGAAGTCTAATCCTTGTGCAACAGGCGGCGCCCTGAAGGGCTATCAGAT
CACCAATAAGTTCGAGAGCTTTAAGTCCATGTCTACCCAGAACGGCTTCATCTTTTA
CATCCCTGCCTGGCTGACATCCAAGATCGATCCATCTACCGGCTTTGTGAACCTGCT
GAAAACCAAGTATACCAGCATCGCCGATTCCAAGAAGTTCATCAGCTCCTTTGACAG
GATCATGTACGTGCCCGAGGAGGATCTGTTCGAGTTTGCCCTGGACTATAAGAACTT
CTCTCGCACAGACGCCGATTACATCAAGAAGTGGAAGCTGTACTCCTACGGCAACC
GGATCAGAATCTTCCGGAATCCTAAGAAGAACAACGTGTTCGACTGGGAGGAGGTG
TGCCTGACCAGCGCCTATAAGGAGCTGTTCAACAAGTACGGCATCAATTATCAGCAG
GGCGATATCAGAGCCCTGCTGTGCGAGCAGTCCGACAAGGCCTTCTACTCTAGCTTT
ATGGCCCTGATGAGCCTGATGCTGCAGATGCGGAACAGCATCACAGGCCGCACCGA
CGTGGATTTTCTGATCAGCCCTGTGAAGAACTCCGACGGCATCTTCTACGATAGCCG
GAACTATGAGGCCCAGGAGAATGCCATCCTGCCAAAGAACGCCGACGCCAATGGCG
CCTATAACATCGCCAGAAAGGTGCTGTGGGCCATCGGCCAGTTCAAGAAGGCCGAG
GACGAGAAGCTGGATAAGGTGAAGATCGCCATCTCTAACAAGGAGTGGCTGGAGTA
CGCCCAGACCAGCGTGAAGCACAAAAGGCCGGCGGCCACGAAAAAGGCCGGCCAG
GCAAAAAAGAAAAAGGGATCCTACCCATACGATGTTCCAGATTACGCTTATCCCTAC
GACGTGCCTGATTATGCATACCCATATGATGTCCCCGACTATGCCTAAGaattctgcagatat
ccagcacagtggcggccgctcgagtctagagggcccgtttaaacccgctgatcagcctcgactgtgccttctagttgccagccatctgttgtt
tgcccctcccccgtgccttccttgaccctggaaggtgccactcccactgtcctttcctaataaaatgaggaaattgcatcgcattgtctgagtag
gtgtcattctattctggggggtggggtggggcaggacagcaagggggaggattgggaagacaatagcaggcatgctggggatgcggtg
ggctctatggcttctgaggcggaaagaaccagctggggctctagggggtatccccacgcgccctgtagcggcgcattaagcgcggcggg
tgtggtggttacgcgcagcgtgaccgctacacttgccagcgccctagcgcccgctcctttcgctttcttcccttcctttctcgccacgttcgccg
gctttccccgtcaagctctaaatcgggggctccctttagggttccgatttagtgctttacggcacctcgaccccaaaaaacttgattagggtgat
ggttcacgtagtgggccatcgccctgatagacggtttttcgccctttgacgttggagtccacgttctttaatagtggactcttgttccaaactgg
aacaacactcaaccctatctcggtctattcttttgatttataagggattttgccgatttcggcctattggttaaaaaatgagctgatttaacaaa
aatttaacgcgaattaattctgtggaatgtgtgtcagttagggtgtggaaagtccccaggctccccagcaggcagaagtatgcaaagcatgcatc
tcaattagtcagcaaccaggtgtggaaagtccccaggctccccagcaggcagaagtatgcaaagcatgcatctcaattagtcagcaaccatag
tcccgcccctaactccgcccatcccgcccctaactccgcccagttccgcccattctccgccccatggctgactaattttttttatttatgcagag
gccgaggccgcctctgcctctgagctattccagaagtagtgaggaggcttttttggaggcctaggcttttgcaaaaagctcccgggagcttgt
atatccattttcggatctgatcaagagacaggatgaggatcgtttcgcatgattgaacaagatggattgcacgcaggttctccggccgcttgg
gtggagaggctattcggctatgactgggcacaacagacaatcggctgctctgatgccgccgtgttccggctgtcagcgcaggggcgcccg
gttctttttgtcaagaccgacctgtccggtgccctgaatgaactgcaggacgaggcagcgcggctatcgtggctggccacgacgggcgttc
cttgcgcagctgtgctcgacgttgtcactgaagcgggaagggactggctgctattgggcgaagtgccggggcaggatctcctgtcatctca
ccttgctcctgccgagaaagtatccatcatggctgatgcaatgcggcggctgcatacgcttgatccggctacctgcccattcgaccaccaag
cgaaacatcgcatcgagcgagcacgtactcggatggaagccggtcttgtcgatcaggatgatctggacgaagagcatcaggggctcgcg
ccagccgaactgttcgccaggctcaaggcgcgcatgcccgacggcgaggatctcgtcgtgacccatggcgatgcctgcttgccgaatatc
atggtggaaaatggccgcttttctggattcatcgactgtggccggctgggtgtggcggaccgctatcaggacatagcgttggctacccgtga
tattgctgaagagcttggcggcgaatgggctgaccgcttcctcgtgctttacggtatcgccgctcccgattcgcagcgcatcgccttctatcg
ccttcttgacgagttcttctgagcgggactctggggttcgaaatgaccgaccaagcgacgcccaacctgccatcacgagatttcgattccac
cgccgccttctatgaaaggttgggcttcggaatcgttttccgggacgccggctggatgatcctccagcgcggggatctcatgctggagttctt
cgcccaccccaacttgtttattgcagcttataatggttacaaataaagcaatagcatcacaaatttcacaaataaagcatttttttcactgcatt
ctagttgtggtttgtccaaactcatcaatgtatcttatcatgtctgtataccgtcgacctctagctagagcttggcgtaatcatggtcatagctg
tttcctgtgtgaaattgttatccgctcacaattccacacaacatacgagccggaagcataaagtgtaaagcctggggtgcctaatgagtgagcta
actcacattaattgcgttgcgctcactgcccgctttccagtcgggaaacctgtcgtgccagctgcattaatgaatcggccaacgcgcggggagag
gcggtttgcgtattgggcgctcttccgcttcctcgctcactgactcgctgcgctcggtcgttcggctgcggcgagcggtatcagctcactcaa
aggcggtaatacggttatccacagaatcaggggataacgcaggaaagaacatgtgagcaaaaggccagcaaaaggccaggaaccgtaa
aaaggccgcgttgctggcgtttttccataggctccgcccccctgacgagcatcacaaaaatcgacgctcaagtcagaggtggcgaaaccc
gacaggactataaagataccaggcgtttccccctggaagctccctcgtgcgctctcctgttccgaccctgccgcttaccggatacctgtccgc
ctttctcccttcgggaagcgtggcgctttctcatagctcacgctgtaggtatctcagttcggtgtaggtcgttcgctccaagctgggctgtgtgc
acgaaccccccgttcagcccgaccgctgcgccttatccggtaactatcgtcttgagtccaacccggtaagacacgacttatcgccactggca
gcagccactggtaacaggattagcagagcgaggtatgtaggcggtgctacagagttcttgaagtggtggcctaactacggctacactagaa
gaacagtatttggtatctgcgctctgctgaagccagttaccttcggaaaaagagttggtagctcttgatccggcaaacaaaccaccgctggta
gcggtttttttgtttgcaagcagcagattacgcgcagaaaaaaaggatctcaagaagatcctttgatcttttctacggggtctgacgctcagtgg
aacgaaaactcacgttaagggattttggtcatgagattatcaaaaaggatcttcacctagatccttttaaattaaaaatgaagttttaaatcaat
ctaaagtatatatgagtaaacttggtctgacagttaccaatgcttaatcagtgaggcacctatctcagcgatctgtctatttcgttcatccatag
ttgcctgactccccgtcgtgtagataactacgatacgggagggcttaccatctggccccagtgctgcaatgataccgcgagacccacgctcaccg
gctccagatttatcagcaataaaccagccagccggaagggccgagcgcagaagtggtcctgcaactttatccgcctccatccagtctattaa
ttgttgccgggaagctagagtaagtagttcgccagttaatagtttgcgcaacgttgttgccattgctacaggcatcgtggtgtcacgctcgtcgt
ttggtatggcttcattcagctccggttcccaacgatcaaggcgagttacatgatcccccatgttgtgcaaaaaagcggttagctccttcggtcct
ccgatcgttgtcagaagtaagttggccgcagtgttatcactcatggttatggcagcactgcataattctcttactgtcatgccatccgtaagatg
cttttctgtgactggtgagtactcaaccaagtcattctgagaatagtgtatgcggcgaccgagttgctcttgcccggcgtcaatacgggataat
accgcgccacatagcagaactttaaaagtgctcatcattggaaaacgttcttcggggcgaaaactctcaaggatcttaccgctgttgagatcc
agttcgatgtaacccactcgtgcacccaactgatcttcagcatcttttactttcaccagcgtttctgggtgagcaaaaacaggaaggcaaaatg
ccgcaaaaaagggaataagggcgacacggaaatgttgaatactcatactcttcctttttcaatattattgaagcatttatcagggttattgtctc
atgagcggatacatatttgaatgtatttagaaaaataaacaaataggggttccgcgcacatttccccgaaaagtgccacctgacgtc.

A Cpf1 endonuclease can interact with one or more Cpf1 gRNAs and, in concert with the Cpf1 gRNA(s), localizes to a site which comprises a target domain, and in certain embodiments, a PAM sequence. In certain embodiments, the ability of a Cpf1 endonuclease to interact with and cleave a target nucleic acid is PAM sequence dependent. A PAM sequence is a sequence in the target nucleic acid. In certain embodiments, cleavage of the target nucleic acid occurs upstream from the PAM sequence. Cpf1 endonucleases from different bacterial species can recognize different sequence motifs (e.g., PAM sequences). In certain embodiments, a Cpf1 endonuclease recognizes a PAM of TTTA (SEQ ID NO: 120), TTTG (SEQ ID NO: 121), TTTC (SEQ ID NO: 122), or TTTT (SEQ ID NO: 123).

In certain embodiments, the vector encodes at least one Cpf1 endonuclease that recognizes a PAM of TTTA (SEQ ID NO: 120), TTTG (SEQ ID NO: 121), TTTC (SEQ ID NO: 122), or TTTT (SEQ ID NO: 123). In certain embodiments, the at least one Cpf1 endonuclease is a Cpf1 endonuclease from Lachospiraceae bacterium ND2006 (“LbCpf1”) or from Acidaminococcus (“AsCpf1”). In certain embodiments, the Cpf1 endonuclease is encoded by the polynucleotide sequence of SEQ ID NO: 124 or SEQ ID NO: 125.

A nucleic acid encoding a Cpf1 endonuclease can be a synthetic nucleic acid sequence. For example, the synthetic nucleic acid molecule can be chemically modified. The synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein.

Additionally or alternatively, a nucleic acid encoding a Cpf1 endonuclease may comprise a nuclear localization sequence (NLS). Nuclear localization sequences are known in the art.

b. Cpf1 gRNAs

The CRISPR/Cpf1-based gene editing system includes at least one Cpf1 gRNA, e.g., one Cpf1 gRNA, two Cpf1 gRNAs, three gRNAs, etc. The gRNA provides the targeting of a CRISPR/Cpf1-based gene editing system. The Cpf1 gRNA may target any desired DNA sequence by exchanging the sequence encoding a protospacer which confers targeting specificity with the desired DNA target. The “target region”, “target sequence” or “protospacer” as used interchangeably herein refers to the region of the target gene (e.g., a dystrophin gene) to which the CRISPR/Cpf1-based gene editing system targets. The target sequence or protospacer is preceded by a PAM sequence at the 5′ end of the protospacer. In some embodiments, the PAM sequence may be TTTA (SEQ ID NO: 120), TTTG (SEQ ID NO: 121), TTTC (SEQ ID NO: 122), or TTTT (SEQ ID NO: 123).

In some embodiments, the protospacer can be between about 17 bp to about 23 bp. In some embodiments, the Cpf1 gRNA can include a polynucleotide sequence that corresponds to the protospacer or a fragment thereof. In some embodiments, the Cpf1 gRNA can include between about 17 bp to about 23 bp of the protospacer. In some embodiments, the about 17 bp to about 23 bp of the protospacer are consecutive.

In some embodiments, the target region can include a polynucleotide sequence of any one of SEQ ID NOs: 1-35, a fragment of any one of SEQ ID NOs: 1-35, or complement thereof. In some embodiments, the Cpf1 gRNA includes a polynucleotide sequence of any one of SEQ ID NOs: 36-119, a fragment of any one of SEQ ID NOs: 36-119, or complement thereof. In some embodiments, the fragment of any one of SEQ ID NOs: 36-119 is about 17 bp to about 23 bp in length. In some embodiments, the about 17 bp to about 23 bp in the fragment are consecutive.

The CRISPR/Cpf1-based gene editing system may include at least one Cpf1 gRNA, wherein the gRNAs target different DNA sequences. The target DNA sequences may be overlapping. The number of Cpf1 gRNAs encoded by a presently disclosed genetic construct (e.g., an AAV vector) can be at least 1 Cpf1 gRNA, at least 2 different Cpf1 gRNA, at least 3 different Cpf1 gRNA at least 4 different Cpf1 gRNA, at least 5 different Cpf1 gRNA, at least 6 different Cpf1 gRNA, at least 7 different Cpf1 gRNA, at least 8 different Cpf1 gRNA, at least 9 different Cpf1 gRNA, at least 10 different Cpf1 gRNAs, at least 11 different Cpf1 gRNAs, at least 12 different Cpf1 gRNAs, at least 13 different Cpf1 gRNAs, at least 14 different Cpf1 gRNAs, at least 15 different Cpf1 gRNAs, at least 16 different Cpf1 gRNAs, at least 17 different Cpf1 gRNAs, at least 18 different Cpf1 gRNAs, at least 18 different Cpf1 gRNAs, at least 20 different Cpf1 gRNAs, at least 25 different Cpf1 gRNAs, at least 30 different Cpf1 gRNAs, at least 35 different Cpf1 gRNAs, at least 40 different Cpf1 gRNAs, at least 45 different Cpf1 gRNAs, or at least 50 different Cpf1 gRNAs. The number of Cpf1 gRNA encoded by a presently disclosed vector can be between at least 1 Cpf1 gRNA to at least 50 different Cpf1 gRNAs, at least 1 Cpf1 gRNA to at least 45 different Cpf1 gRNAs, at least 1 Cpf1 gRNA to at least 40 different Cpf1 gRNAs, at least 1 Cpf1 gRNA to at least 35 different Cpf1 gRNAs, at least 1 Cpf1 gRNA to at least 30 different Cpf1 gRNAs, at least 1 Cpf1 gRNA to at least 25 different Cpf1 gRNAs, at least 1 Cpf1 gRNA to at least 20 different Cpf1 gRNAs, at least 1 Cpf1 gRNA to at least 16 different Cpf1 gRNAs, at least 1 Cpf1 gRNA to at least 12 different Cpf1 gRNAs, at least 1 Cpf1 gRNA to at least 8 different Cpf1 gRNAs, at least 1 Cpf1 gRNA to at least 4 different Cpf1 gRNAs, at least 4 Cpf1 gRNAs to at least 50 different Cpf1 gRNAs, at least 4 different Cpf1 gRNAs to at least 45 different Cpf1 gRNAs, at least 4 different Cpf1 gRNAs to at least 40 different Cpf1 gRNAs, at least 4 different Cpf1 gRNAs to at least 35 different Cpf1 gRNAs, at least 4 different Cpf1 gRNAs to at least 30 different Cpf1 gRNAs, at least 4 different Cpf1 gRNAs to at least 25 different Cpf1 gRNAs, at least 4 different Cpf1 gRNAs to at least 20 different Cpf1 gRNAs, at least 4 different Cpf1 gRNAs to at least 16 different Cpf1 gRNAs, at least 4 different Cpf1 gRNAs to at least 12 different Cpf1 gRNAs, at least 4 different Cpf1 gRNAs to at least 8 different Cpf1 gRNAs, at least 8 different Cpf1 gRNAs to at least 50 different Cpf1 gRNAs, at least 8 different Cpf1 gRNAs to at least 45 different Cpf1 gRNAs, at least 8 different Cpf1 gRNAs to at least 40 different Cpf1 gRNAs, at least 8 different Cpf1 gRNAs to at least 35 different Cpf1 gRNAs, 8 different Cpf1 gRNAs to at least 30 different Cpf1 gRNAs, at least 8 different Cpf1 gRNAs to at least 25 different Cpf1 gRNAs, 8 different Cpf1 gRNAs to at least 20 different Cpf1 gRNAs, at least 8 different Cpf1 gRNAs to at least 16 different Cpf1 gRNAs, or 8 different Cpf1 gRNAs to at least 12 different Cpf1 gRNAs. In certain embodiments, the genetic construct (e.g., an AAV vector) encodes one Cpf1 gRNA, i.e., a first Cpf1 gRNA, and optionally a Cpf1 endonuclease. In certain embodiments, a first genetic construct (e.g., a first AAV vector) encodes one Cpf1 gRNA, i.e., a first Cpf1 gRNA, and optionally a Cpf1 endonuclease, and a second genetic construct (e.g., a second AAV vector) encodes one Cpf1 gRNA, i.e., a second Cpf1 gRNA, and optionally a Cpf1 endonuclease.

### 3. CRISPR/Cpf1-Based Gene Editing System Genetic Constructs for Genome Editing of Dystrophin Gene

The present invention is directed to genetic constructs for genome editing, genomic alteration or altering gene expression of a dystrophin gene (e.g., human dystrophin gene). The genetic constructs include at least one Cpf1 gRNA that targets human dystrophin gene sequences, such as Cpf1 endonuclease-compatible targets. The disclosed gRNAs can be included in a CRISPR/Cpf1-based gene editing system, including systems that use Cpf1 endonuclease, to target regions in the dystrophin gene, such as intronic regions surrounding exons, such as exon 51, of the human dystrophin gene, splice acceptor sites, and/or exonic regions, causing genomic deletions of this region in order to restore expression of functional dystrophin in cells from DMD patients.

DMD is a severe muscle wasting disease caused by genetic mutations to the dystrophin gene. Dystrophin is a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane. The dystrophin gene is 2.2 megabases at locus Xp21. The primary transcription measures about 2,400 kb with the mature mRNA being about 14 kb. 79 exons code for the protein which is over 3500 amino acids. Normal skeleton muscle tissue contains only small amounts of dystrophin but its absence of abnormal expression leads to the development of severe and incurable symptoms. Some mutations in the dystrophin gene lead to the production of defective dystrophin and severe dystrophic phenotype in affected patients. Some mutations in the dystrophin gene lead to partially-functional dystrophin protein and a much milder dystrophic phenotype in affected patients.

DMD is the result of inherited or spontaneous mutations that cause nonsense or frame shift mutations in the dystrophin gene. Naturally occurring mutations and their consequences are relatively well understood for DMD. Mutations are typically deletions or duplications of regions of the gene that make the protein out of frame and completely dysfunctional. Removal of single exons can be applied to as many as 83% of patients by frame corrections restoring a nearly functional protein. CPF1 can target dystrophin exons and be used to knockout single exons by targeting splice acceptors or delete genetic regions to remove single or multiple exons.

It is known that in-frame deletions that occur in the exon 45-55 regions (e.g., exon 51) contained within the rod domain can produce highly functional dystrophin proteins, and many carriers are asymptomatic or display mild symptoms. Furthermore, more than 60% of patients may theoretically be treated by targeting exons in this region of the dystrophin gene (e.g., targeting an exon of dystrophin gene, such as exon 51). Efforts have been made to restore the disrupted dystrophin reading frame in DMD patients by skipping non-essential exon(s) (e.g., exon 51 skipping) during mRNA splicing to produce internally deleted but functional dystrophin proteins. The deletion of internal dystrophin exon(s) (e.g., deletion of exon 51) retains the proper reading frame but cause the less severe Becker muscular dystrophy, or BMD. The Becker muscular dystrophy, or BMD, genotype is similar to DMD in that deletions are present in the dystrophin gene. However, these deletions leave the reading frame intact. Thus an internally truncated but partially functional dystrophin protein is created. BMD has a wide array of phenotypes, but often if deletions are between exons 45-55 of dystrophin the phenotype is much milder compared to DMD. Thus changing a DMD genotype to a BMD genotype is a common strategy to correct dystrophin. There are many strategies to correct dystrophin, many of which rely on restoring the reading frame of the endogenous dystrophin. This shifts the disease genotype from DMD to Becker muscular dystrophy. Many BMD patients have intragenic deletions that maintain the translational reading frame, leading to a shorter but largely functional dystrophin protein.

In certain embodiments, modification of exon 51 (e.g., deletion or excision of exon 51 by, e.g., NHEJ) to restore reading frame ameliorates the phenotype DMD subjects, including DMD subjects with deletion mutations. In certain embodiments, exon 51 of a dystrophin gene refers to the 51^{st} exon of the dystrophin gene. Exon 51 is frequently adjacent to frame-disrupting deletions in DMD patients and has been targeted in clinical trials for oligonucleotide-based exon skipping. A clinical trial for the exon 51 skipping compound eteplirsen reported a significant functional benefit across 48 weeks, with an average of 47% dystrophin positive fibers compared to baseline. Mutations in exon 51 are ideally suited for permanent correction by NHEJ-based genome editing.

The presently disclosed vectors can generate deletions in the dystrophin gene, e.g., the human dystrophin gene. In certain embodiments, the vector is configured to form two double stand breaks (a first double strand break and a second double strand break) in two introns (a first intron and a second intron) flanking a target position of the dystrophin gene, thereby deleting a segment of the dystrophin gene comprising the dystrophin target position. A “dystrophin target position” can be a dystrophin exonic target position or a dystrophin intra-exonic target position, as described herein. Deletion of the dystrophin exonic target position can optimize the dystrophin sequence of a subject suffering from Duchenne muscular dystrophy, e.g., it can increase the function or activity of the encoded dystrophin protein, or results in an improvement in the disease state of the subject. In certain embodiments, excision of the dystrophin exonic target position restores reading frame. The dystrophin exonic target position can comprise one or more exons of the dystrophin gene. In certain embodiments, the dystrophin target position comprises exon 51 of the dystrophin gene (e.g., human dystrophin gene).

A presently disclosed genetic construct (e.g., a vector) can mediate highly efficient gene editing at exon 51 of a dystrophin gene (e.g., the human dystrophin gene). A presently disclosed genetic construct (e.g., a vector) can restore dystrophin protein expression in cells from DMD patients. Exon 51 is frequently adjacent to frame-disrupting deletions in DMD. Elimination of exon 51 from the dystrophin transcript by exon skipping can be used to treat approximately 15% of all DMD patients. This class of dystrophin mutations is ideally suited for permanent correction by NHEJ-based genome editing and HDR. The genetic constructs (e.g., vectors) described herein have been developed for targeted modification of exon 51 in the human dystrophin gene. A presently disclosed genetic construct (e.g., a vector) is transfected into human DMD cells and mediates efficient gene modification and conversion to the correct reading frame. Protein restoration is concomitant with frame restoration and detected in a bulk population of CRISPR/Cpf1-based gene editing system-treated cells.

Single or multiplexed gRNAs can be designed to restore the dystrophin reading frame by targeting the mutational hotspot at exon 51 or and introducing either intraexonic small insertions and deletions, or excision of exon 51. Following treatment with a presently disclosed vector, dystrophin expression can be restored in Duchenne patient muscle cells in vitro. Human dystrophin was detected in vivo following transplantation of genetically corrected patient cells into immunodeficient mice. Significantly, the unique multiplex gene editing capabilities of the CRISPR/Cpf1-based gene editing system enable efficiently generating large deletions of this mutational hotspot region that can correct up to 62% of patient mutations by universal or patient-specific gene editing approaches. In some embodiments, candidate gRNAs are evaluated and chosen based on off-target activity, on-target activity as measured by surveyor, and distance from the exon.

The Cpf1 gRNA may target a region of the dystrophin gene (DMD). In certain embodiments, the Cpf1 gRNA can target at least one of exons, introns, the promoter region, the enhancer region, splice acceptor sites, and/or the transcribed region of the dystrophin gene. In some embodiments, the target region comprises a polynucleotide sequence of at least one of SEQ ID NOs: 1-28. In certain embodiments, the Cpf1 gRNA targets intron 50 of the human dystrophin gene. In certain embodiments, the Cpf1 gRNA targets intron 51 of the human dystrophin gene. In certain embodiments, the Cpf1 gRNA targets exon 51 of the human dystrophin gene. The Cpf1 gRNA may include a polynucleotide sequence of any one of SEQ ID NO: 36-64, 71-119, a fragment of any one of SEQ ID NOs: 36-64, 71-119, or a complement thereof.

### 4. CRISPR/Cpf1-Based Gene Editing System Genetic Constructs for Genome Editing of B-Cell Lymphoma/Leukemia 11A (BCL11a) Gene

Sickle cell anemia (SCA) is caused by a point mutation in the β-globin gene, and β-thalassemia is caused by other mutations leading to loss of β-globin expression. BCL11a is a transcriptional repressor that silences embryonic and fetal globin genes. Complete loss of BCL11a is embryonically lethal; however, disrupting the erythroid-specific enhancer region of BCL11a may reduce the abundance of the transcriptional repressor and increase fetal globin levels improving phenotype of the disease. Similarly, a particular mutation to the γ-globin (HBG1/2) promoter leads to loss of transcriptional repression and hereditary persistence of fetal hemoglobin (HPFH). The larger indel footprint generated by Cpf1 can efficiently disrupt the enhancer region of BCL11a or repression regions of HBG1/2. In some embodiments, the Cpf1 gRNAs is designed to disrupt the enhancer region of BCL11a, increase fetal globin levels, and improve phenotype of SCA. In some embodiments, the enhancer region comprises a polynucleotide sequence of at least one of SEQ ID NOs: 29-35. In some embodiments, the Cpf1 gRNA comprises a polynucleotide sequence of any one of SEQ ID NOs: 65-70, a fragment of any one of SEQ ID NOs: 65-70, or a complement thereof.

### 5. DNA Targeting Compositions

The present invention is also directed to DNA targeting compositions that comprise such genetic constructs. The DNA targeting compositions include at least one Cpf1 gRNA (e.g., one Cpf1 gRNA, two Cpf1 gRNAs, three gRNAs, etc.) that targets a dystrophin gene (e.g., human dystrophin gene), as described above. The at least one Cpf1 gRNA can bind and recognize a target region. The target regions can be chosen immediately upstream of possible out-of-frame stop codons such that insertions or deletions during the repair process restore the dystrophin reading frame by frame conversion. Target regions can also be splice acceptor sites or splice donor sites, such that insertions or deletions during the repair process disrupt splicing and restore the dystrophin reading frame by splice site disruption and exon exclusion. Target regions can also be aberrant stop codons such that insertions or deletions during the repair process restore the dystrophin reading frame by eliminating or disrupting the stop codon.

In certain embodiments, the presently disclosed DNA targeting composition includes a first Cpf1 gRNA and a second Cpf1 gRNA, wherein the first Cpf1 gRNA and the second Cpf1 gRNA comprise a polynucleotide sequence set forth in SEQ ID NOs: 36-119, or a complement thereof. In some embodiments the polynucleotide sequence comprises at least one of SEQ ID NOs: 36-64, 71-119, or a complement thereof. In some embodiments the polynucleotide sequence comprises at least one of SEQ ID NOs: 65-70, or a complement thereof. In certain embodiments, the first Cpf1 gRNA and the second Cpf1 gRNA comprise polynucleotide sequences.

In certain embodiments, the first Cpf1 gRNA and the second Cpf1 gRNA are selected from the group consisting of: (i) a first Cpf1 gRNA comprising a polynucleotide sequence set forth in SEQ ID NO: 54, and a second Cpf1 gRNA comprising a polynucleotide sequence set forth in SEQ ID NO: 62; (ii) a first Cpf1 gRNA comprising a polynucleotide sequence set forth in SEQ ID NO: 55, and a second Cpf1 gRNA comprising a polynucleotide sequence set forth in SEQ ID NO: 63; and (iii) a first Cpf1 gRNA comprising a polynucleotide sequence set forth in SEQ ID NO: 56, and a second Cpf1 gRNA comprising a polynucleotide sequence set forth in SEQ ID NO: 61.

In certain embodiments, the DNA targeting composition may further include at least one Cpf1 endonuclease that recognizes a PAM of TTTA (SEQ ID NO: 120), TTTG (SEQ ID NO: 121), TTTC (SEQ ID NO: 122), or TTTT (SEQ ID NO: 123). In some embodiments, the DNA targeting composition includes a Cpf1 endonuclease encoded by a polynucleotide sequence set forth in SEQ ID NO: 124 or SEQ ID NO: 125. In certain embodiments, the vector is configured to form a first and a second double strand break in a first and a second intron flanking exon 51 of the human dystrophin gene, respectively, thereby deleting a segment of the dystrophin gene comprising exon 51.

The deletion efficiency of the presently disclosed vectors can be related to the deletion size, i.e., the size of the segment deleted by the vectors. In certain embodiments, the length or size of specific deletions is determined by the distance between the PAM sequences in the gene being targeted (e.g., a dystrophin gene). In certain embodiments, a specific deletion of a segment of the dystrophin gene, which is defined in terms of its length and a sequence it comprises (e.g., exon 51), is the result of breaks made adjacent to specific PAM sequences within the target gene (e.g., a dystrophin gene).

In certain embodiments, the deletion size is about 50 to about 2,000 base pairs (bp), e.g., about 50 to about 1999 bp, about 50 to about 1900 bp, about 50 to about 1800 bp, about 50 to about 1700 bp, about 50 to about 1650 bp, about 50 to about 1600 bp, about 50 to about 1500 bp, about 50 to about 1400 bp, about 50 to about 1300 bp, about 50 to about 1200 bp, about 50 to about 1150 bp, about 50 to about 1100 bp, about 50 to about 1000 bp, about 50 to about 900 bp, about 50 to about 850 bp, about 50 to about 800 bp, about 50 to about 750 bp, about 50 to about 700 bp, about 50 to about 600 bp, about 50 to about 500 bp, about 50 to about 400 bp, about 50 to about 350 bp, about 50 to about 300 bp, about 50 to about 250 bp, about 50 to about 200 bp, about 50 to about 150 bp, about 50 to about 100 bp, about 100 to about 1999 bp, about 100 to about 1900 bp, about 100 to about 1800 bp, about 100 to about 1700 bp, about 100 to about 1650 bp, about 100 to about 1600 bp, about 100 to about 1500 bp, about 100 to about 1400 bp, about 100 to about 1300 bp, about 100 to about 1200 bp, about 100 to about 1150 bp, about 100 to about 1100 bp, about 100 to about 1000 bp, about 100 to about 900 bp, about 100 to about 850 bp, about 100 to about 800 bp, about 100 to about 750 bp, about 100 to about 700 bp, about 100 to about 600 bp, about 100 to about 1000 bp, about 100 to about 400 bp, about 100 to about 350 bp, about 100 to about 300 bp, about 100 to about 250 bp, about 100 to about 200 bp, about 100 to about 150 bp, about 200 to about 1999 bp, about 200 to about 1900 bp, about 200 to about 1800 bp, about 200 to about 1700 bp, about 200 to about 1650 bp, about 200 to about 1600 bp, about 200 to about 1500 bp, about 200 to about 1400 bp, about 200 to about 1300 bp, about 200 to about 1200 bp, about 200 to about 1150 bp, about 200 to about 1100 bp, about 200 to about 1000 bp, about 200 to about 900 bp, about 200 to about 850 bp, about 200 to about 800 bp, about 200 to about 750 bp, about 200 to about 700 bp, about 200 to about 600 bp, about 200 to about 2000 bp, about 200 to about 400 bp, about 200 to about 350 bp, about 200 to about 300 bp, about 200 to about 250 bp, about 300 to about 1999 bp, about 300 to about 1900 bp, about 300 to about 1800 bp, about 300 to about 1700 bp, about 300 to about 1650 bp, about 300 to about 1600 bp, about 300 to about 1500 bp, about 300 to about 1400 bp, about 300 to about 1300 bp, about 300 to about 1200 bp, about 300 to about 1150 bp, about 300 to about 1100 bp, about 300 to about 1000 bp, about 300 to about 900 bp, about 300 to about 850 bp, about 300 to about 800 bp, about 300 to about 750 bp, about 300 to about 700 bp, about 300 to about 600 bp, about 300 to about 3000 bp, about 300 to about 400 bp, or about 300 to about 350 bp. In certain embodiments, the deletion size can be about 118 base pairs, about 233 base pairs, about 326 base pairs, about 766 base pairs, about 805 base pairs, or about 1611 base pairs.

### 6. Compositions for Genome Editing in Muscle

The present invention is directed to genetic constructs (e.g., vectors) or a composition thereof for genome editing a target gene in skeletal muscle or cardiac muscle of a subject. The composition includes a modified AAV vector and a polynucleotide sequence encoding a CRISPR/Cpf1-based gene editing system, e.g., a Cpf1 gRNA and a Cpf1 endonuclease. The composition delivers active forms of CRISPR/Cpf1-based gene editing systems to skeletal muscle or cardiac muscle. The presently disclosed genetic constructs (e.g., vectors) can be used in correcting or reducing the effects of mutations in the dystrophin gene involved in genetic diseases and/or other skeletal or cardiac muscle conditions, e.g., DMD. The composition may further comprise a donor DNA or a transgene. These compositions may be used in genome editing, genome engineering, and correcting or reducing the effects of mutations in genes involved in genetic diseases and/or other skeletal or cardiac muscle conditions.

a. CRISPR/Cpf1-Based Gene Editing System for Targeting Dystrophin

A CRISPR/Cpf1-based gene editing system specific for dystrophin gene are disclosed herein. The CRISPR/Cpf1-based gene editing system may include Cpf1 endonuclease and at least one Cpf1 gRNA to target the dystrophin gene. The CRISPR/Cpf1-based gene editing system may bind and recognize a target region. The target regions may be chosen immediately upstream of possible out-of-frame stop codons such that insertions or deletions during the repair process restore the dystrophin reading frame by frame conversion. Target regions may also be splice acceptor sites or splice donor sites, such that insertions or deletions during the repair process disrupt splicing and restore the dystrophin reading frame by splice site disruption and exon exclusion. Target regions may also be aberrant stop codons such that insertions or deletions during the repair process restore the dystrophin reading frame by eliminating or disrupting the stop codon.

The Cpf1 gRNA may target a polynucleotide sequence selected from the group consisting of SEQ ID NO: 1-35, or a complement thereof. For example, the disclosed CRISPR/Cpf1-based gene editing systems were engineered to mediate highly efficient gene editing at exon 51 of the dystrophin gene. These CRISPR/Cpf1-based gene editing systems restored dystrophin protein expression in cells from DMD patients.

b. Adeno-Associated Virus Vectors

The composition may also include a viral delivery system. In certain embodiments, the vector is an adeno-associated virus (AAV) vector. The AAV vector is a small virus belonging to the genus Dependovirus of the Parvoviridae family that infects humans and some other primate species. AAV vectors may be used to deliver CRISPR/Cpf1-based gene editing systems using various construct configurations. For example, AAV vectors may deliver Cpf1 endonucleases and Cpf1 gRNA expression cassettes on separate vectors or on the same vector. Alternatively, both the Cpf1 endonucleases and up to two gRNA expression cassettes may be combined in a single AAV vector within the 4.7 kb packaging limit.

In certain embodiments, the AAV vector is a modified AAV vector. The modified AAV vector may have enhanced cardiac and skeletal muscle tissue tropism. The modified AAV vector may be capable of delivering and expressing the CRISPR/Cpf1-based gene editing system in the cell of a mammal. For example, the modified AAV vector may be an AAV-SASTG vector (Piacentino et al. (2012) Human Gene Therapy 23:635-646). The modified AAV vector may deliver nucleases to skeletal and cardiac muscle in vivo. The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5 and AAV/SASTG vectors that efficiently transduce skeletal muscle or cardiac muscle by systemic and local delivery (Seto et al. Current Gene Therapy (2012) 12:139-151). The modified AAV vector may be AAV2i8G9 (Shen et al. J. Biol. Chem. (2013) 288:28814-28823).

### 7. Methods of Genome Editing in Muscle

The present disclosure is directed to a method of genome editing in a skeletal muscle or cardiac muscle of a subject. The method comprises administering to the skeletal muscle or cardiac muscle of the subject the composition for genome editing in skeletal muscle or cardiac muscle, as described above. The genome editing may include correcting a mutant gene or inserting a transgene. Correcting the mutant gene may include deleting, rearranging, or replacing the mutant gene. Correcting the mutant gene may include nuclease-mediated NHEJ or HDR.

### 8. Methods of Correcting a Mutant Gene and Treating a Subject

The presently disclosed subject matter provides for methods of correcting a mutant gene (e.g., a mutant dystrophin gene, e.g., a mutant human dystrophin gene) in a cell and treating a subject suffering from a genetic disease, such as DMD. The method can include administering to a cell or a subject a presently disclosed genetic construct (e.g., a vector) or a composition comprising thereof as described above. The method can comprises administering to the skeletal muscle or cardiac muscle of the subject the presently disclosed genetic construct (e.g., a vector) or a composition comprising thereof for genome editing in skeletal muscle or cardiac muscle, as described above. Use of presently disclosed genetic construct (e.g., a vector) or a composition comprising thereof to deliver the CRISPR/Cpf1-based gene editing system to the skeletal muscle or cardiac muscle may restore the expression of a full-functional or partially-functional protein with a repair template or donor DNA, which can replace the entire gene or the region containing the mutation. The CRISPR/Cpf1-based gene editing system may be used to introduce site-specific double strand breaks at targeted genomic loci. Site-specific double-strand breaks are created when the CRISPR/Cpf1-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

The present disclosure is directed to genome editing with a CRISPR/Cpf1-based gene editing system without a repair template, which can efficiently correct the reading frame and restore the expression of a functional protein involved in a genetic disease. The disclosed CRISPR/Cpf1-based gene editing systems may involve using homology-directed repair or nuclease-mediated non-homologous end joining (NHEJ)-based correction approaches, which enable efficient correction in proliferation-limited primary cell lines that may not be amenable to homologous recombination or selection-based gene correction. This strategy integrates the rapid and robust assembly of active CRISPR/Cpf1-based gene editing systems with an efficient gene editing method for the treatment of genetic diseases caused by mutations in nonessential coding regions that cause frameshifts, premature stop codons, aberrant splice donor sites or aberrant splice acceptor sites.

a. Nuclease Mediated Non-Homologous End Joining

Restoration of protein expression from an endogenous mutated gene may be through template-free NHEJ-mediated DNA repair. In contrast to a transient method targeting the target gene RNA, the correction of the target gene reading frame in the genome by a transiently expressed CRISPR/Cpf1-based gene editing system may lead to permanently restored target gene expression by each modified cell and all of its progeny. In certain embodiments, NHEJ is a nuclease mediated NHEJ, which in certain embodiments, refers to NHEJ that is initiated a Cpf1 endonuclease, cuts double stranded DNA. The method comprises administering a presently disclosed genetic construct (e.g., a vector) or a composition comprising thereof to the skeletal muscle or cardiac muscle of the subject for genome editing in skeletal muscle or cardiac muscle.

Nuclease mediated NHEJ gene correction may correct the mutated target gene and offers several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment. NHEJ-based gene correction using a CRISPR/Cpf1-based gene editing system, as well as other engineered nucleases including meganucleases and zinc finger nucleases, may be combined with other existing ex vivo and in vivo platforms for cell- and gene-based therapies, in addition to the plasmid electroporation approach described here. For example, delivery of a CRISPR/Cpf1-based gene editing system by mRNA-based gene transfer or as purified cell permeable proteins could enable a DNA-free genome editing approach that would circumvent any possibility of insertional mutagenesis.

b. Homology-Directed Repair

Restoration of protein expression from an endogenous mutated gene may involve homology-directed repair. The method as described above further includes administrating a donor template to the cell. The donor template may include a polynucleotide sequence encoding a full-functional protein or a partially-functional protein. For example, the donor template may include a miniaturized dystrophin construct, termed minidystrophin (“minidys”), a full-functional dystrophin construct for restoring a mutant dystrophin gene, or a fragment of the dystrophin gene that after homology-directed repair leads to restoration of the mutant dystrophin gene.

c. Methods of Correcting a Mutant Gene and Treating a Subject Using CRISPR/Cpf1-Based Gene Editing System

The present disclosure is also directed to genome editing with the CRISPR/Cpf1-based gene editing system to restore the expression of a full-functional or partially-functional protein with a repair template or donor DNA, which can replace the entire gene or the region containing the mutation. The CRISPR/Cpf1-based gene editing system may be used to introduce site-specific double strand breaks at targeted genomic loci. Site-specific double-strand breaks are created when the CRISPR/Cpf1-based gene editing system binds to a target DNA sequences using the gRNA, thereby permitting cleavage of the target DNA. The CRISPR/Cpf1-based gene editing system has the advantage of advanced genome editing due to their high rate of successful and efficient genetic modification. This DNA cleavage may stimulate the natural DNA-repair machinery, leading to one of two possible repair pathways: homology-directed repair (HDR) or the non-homologous end joining (NHEJ) pathway. For example, a CRISPR/Cpf1-based gene editing system directed towards the dystrophin gene may include a Cpf1 gRNA having a nucleic acid sequence of any one of SEQ ID NOs: 36-64, 71-119, or complement thereof.

The present disclosure is directed to genome editing with CRISPR/Cpf1-based gene editing system without a repair template, which can efficiently correct the reading frame and restore the expression of a functional protein involved in a genetic disease. The disclosed CRISPR/Cpf1-based gene editing system and methods may involve using homology-directed repair or nuclease-mediated non-homologous end joining (NHEJ)-based correction approaches, which enable efficient correction in proliferation-limited primary cell lines that may not be amenable to homologous recombination or selection-based gene correction. This strategy integrates the rapid and robust assembly of active CRISPR/Cpf1-based gene editing system with an efficient gene editing method for the treatment of genetic diseases caused by mutations in nonessential coding regions that cause frameshifts, premature stop codons, aberrant splice donor sites or aberrant splice acceptor sites.

The present disclosure provides methods of correcting a mutant gene in a cell and treating a subject suffering from a genetic disease, such as DMD. The method may include administering to a cell or subject a CRISPR/Cpf1-based gene editing system, a polynucleotide or vector encoding said CRISPR/Cpf1-based gene editing system, or composition of said CRISPR/Cpf1-based gene editing system as described above. The method may include administering a CRISPR/Cpf1-based gene editing system, such as administering a Cpf1 endonuclease, a polynucleotide sequence encoding said Cpf1 endonuclease, and/or at least one Cpf1 gRNA, wherein the gRNAs target different DNA sequences. The target DNA sequences may be overlapping. The number of gRNA administered to the cell may be at least 1 gRNA, at least 2 different gRNA, at least 3 different gRNA at least 4 different gRNA, at least 5 different gRNA, at least 6 different gRNA, at least 7 different gRNA, at least 8 different gRNA, at least 9 different gRNA, at least 10 different gRNA, at least 15 different gRNA, at least 20 different gRNA, at least 30 different gRNA, or at least 50 different gRNA, as described above. The gRNA may include a nucleic acid sequence of at least one of SEQ ID NOs: 36-64, 71-119, or complement thereof. The method may involve homology-directed repair or non-homologous end joining.

### 9. Methods of Treating Disease

The present disclosure is directed to a method of treating a subject in need thereof. The method comprises administering to a tissue of a subject the presently disclosed genetic construct (e.g., a vector) or a composition comprising thereof, as described above. In certain embodiments, the method may comprises administering to the skeletal muscle or cardiac muscle of the subject the presently disclosed genetic construct (e.g., a vector) or composition comprising thereof, as described above. In certain embodiments, the method may comprises administering to a vein of the subject the presently disclosed genetic construct (e.g., a vector) or composition comprising thereof, as described above. In certain embodiments, the subject is suffering from a skeletal muscle or cardiac muscle condition causing degeneration or weakness or a genetic disease. For example, the subject may be suffering from Duchenne muscular dystrophy, as described above.

a. Duchenne Muscular Dystrophy

The method, as described above, may be used for correcting the dystrophin gene and recovering full-functional or partially-functional protein expression of said mutated dystrophin gene. In some aspects and embodiments the disclosure provides a method for reducing the effects (e.g., clinical symptoms/indications) of DMD in a patient. In some aspects and embodiments the disclosure provides a method for treating DMD in a patient. In some aspects and embodiments the disclosure provides a method for preventing DMD in a patient. In some aspects and embodiments the disclosure provides a method for preventing further progression of DMD in a patient.

### 10. Constructs and Plasmids

The compositions, as described above, may comprise genetic constructs that encodes the CRISPR/Cpf1-based gene editing system, as disclosed herein. The genetic construct, such as a plasmid, may comprise a nucleic acid that encodes the CRISPR/Cpf1-based gene editing system, such as the Cpf1 endonuclease and/or at least one of the Cpf1 gRNAs. The compositions, as described above, may comprise genetic constructs that encodes the modified AAV vector and a nucleic acid sequence that encodes the CRISPR/Cpf1-based gene editing system, as disclosed herein. The genetic construct, such as a plasmid, may comprise a nucleic acid that encodes the CRISPR/Cpf1-based gene editing system. The compositions, as described above, may comprise genetic constructs that encodes the modified lentiviral vector, as disclosed herein.

In some embodiments, the genetic construct may comprise a promoter that operably linked to the polynucleotide sequence encoding the at least one Cpf1 gRNA and/or a Cpf1 endonuclease. In some embodiments, the promoter is operably linked to the polynucleotide sequence encoding a first Cpf1 gRNA, a second Cpf1 gRNA, and/or a Cpf1 endonuclease. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant lentivirus, recombinant adenovirus, and recombinant adenovirus associated virus. The genetic construct may be part of the genetic material in attenuated live microorganisms or recombinant microbial vectors which live in cells. The genetic constructs may comprise regulatory elements for gene expression of the coding sequences of the nucleic acid. The regulatory elements may be a promoter, an enhancer, an initiation codon, a stop codon, or a polyadenylation signal.

In certain embodiments, the genetic construct is a vector. The vector can be an Adeno-associated virus (AAV) vector, which encode at least one Cpf1 endonuclease and at least one Cpf1 gRNA; the vector is capable of expressing the at least one Cpf1 endonuclease and the at least one Cpf1 gRNA, in the cell of a mammal. The vector can be a plasmid. The vectors can be used for in vivo gene therapy. The vector may be recombinant. The vector may comprise heterologous nucleic acid encoding the CRISPR/Cpf1-based gene editing system. The vector may be a plasmid. The vector may be useful for transfecting cells with nucleic acid encoding the CRISPR/Cpf1-based gene editing system, which the transformed host cell is cultured and maintained under conditions wherein expression of the CRISPR/Cpf1-based gene editing system takes place.

Coding sequences may be optimized for stability and high levels of expression. In some instances, codons are selected to reduce secondary structure formation of the RNA such as that formed due to intramolecular bonding.

The vector may comprise heterologous nucleic acid encoding the CRISPR/Cpf1-based gene editing system and may further comprise an initiation codon, which may be upstream of the CRISPR/Cpf1-based gene editing system coding sequence, and a stop codon, which may be downstream of the CRISPR/Cpf1-based gene editing system coding sequence. The initiation and termination codon may be in frame with the CRISPR/Cpf1-based gene editing system coding sequence. The vector may also comprise a promoter that is operably linked to the CRISPR/Cpf1-based gene editing system coding sequence. The promoter that is operably linked to the CRISPR/Cpf1-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, a U6 promoter, such as the human U6 promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a muscle or skin specific promoter, natural or synthetic. Examples of such promoters are described in US Patent Application Publication Nos. US20040175727 and US20040192593, the contents of which are incorporated herein in their entirety. Examples of muscle-specific promoters include a Spc5-12 promoter (described in U.S. Patent Application Publication No. U.S. 20040192593, which is incorporated by reference herein in its entirety; Hakim et al. Mol. Ther. Methods Clin. Dev. (2014) 1:14002; and Lai et al. Hum Mol Genet. (2014) 23(12): 3189-3199), a MHCK7 promoter (described in Salva et al., Mol. Ther. (2007) 15:320-329), a CK8 promoter (described in Park et al. PLOS ONE (2015) 10 (4): e0124914), and a CK8e promoter (described in Muir et al., Mol. Ther. Methods Clin. Dev. (2014) 1:14025). In some embodiments, the expression of the gRNA and/or Cpf1 endonuclease is driven by tRNAs.

Each of the polynucleotide sequences encoding the Cpf1 gRNA and/or Cpf1 endonuclease may each be operably linked to a promoter. The promoters that are operably linked to the Cpf1 gRNA and/or Cpf1 endonuclease may be the same promoter. The promoters that are operably linked to the Cpf1 gRNA and/or Cpf1 endonuclease may be different promoters. The promoter may be a constitutive promoter, an inducible promoter, a repressible promoter, or a regulatable promoter.

The vector may also comprise a polyadenylation signal, which may be downstream of the CRISPR/Cpf1-based gene editing system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, CA).

The vector may also comprise an enhancer upstream of the CRISPR/Cpf1-based gene editing system, i.e., the Cpf1 endonuclease coding sequence, Cpf1 gRNAs, or the CRISPR/Cpf1-based gene editing system. The enhancer may be necessary for DNA expression. The enhancer may be human actin, human myosin, human hemoglobin, human muscle creatine or a viral enhancer such as one from CMV, HA, RSV or EBV. Polynucleotide function enhancers are described in U.S. Pat. Nos. 5,593,972, 5,962,428, and WO94/016737, the contents of each are fully incorporated by reference. The vector may also comprise a mammalian origin of replication in order to maintain the vector extrachromosomally and produce multiple copies of the vector in a cell. The vector may also comprise a regulatory sequence, which may be well suited for gene expression in a mammalian or human cell into which the vector is administered. The vector may also comprise a reporter gene, such as green fluorescent protein (“GFP”) and/or a selectable marker, such as hygromycin (“Hygro”).

The vector may be expression vectors or systems to produce protein by routine techniques and readily available starting materials including Sambrook et al., Molecular Cloning and Laboratory Manual, Second Ed., Cold Spring Harbor (1989), which is incorporated fully by reference. In some embodiments the vector may comprise the nucleic acid sequence encoding the CRISPR/Cpf1-based gene editing system, including the nucleic acid sequence encoding the Cpf1 endonuclease and the nucleic acid sequence encoding the at least one Cpf1 gRNA comprising the nucleic acid sequence of at least one of SEQ ID NOs: 36-119, or complement thereof.

### 11. Pharmaceutical Compositions

The presently disclosed subject matter provides for compositions comprising the above-described genetic constructs. The pharmaceutical compositions according to the present invention can be formulated according to the mode of administration to be used. In cases where pharmaceutical compositions are injectable pharmaceutical compositions, they are sterile, pyrogen free and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the composition for genome editing in skeletal muscle or cardiac muscle at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the composition may also include a transfection facilitating agent such as lipids, liposomes, including lecithin liposomes or other liposomes known in the art, as a DNA-liposome mixture (see for example International Patent Publication No. WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

### 12. Methods of Delivery

Provided herein is a method for delivering the presently disclosed genetic construct (e.g., a vector) or a composition thereof to a cell. The delivery of the compositions may be the transfection or electroporation of the composition as a nucleic acid molecule that is expressed in the cell and delivered to the surface of the cell. The nucleic acid molecules may be electroporated using BioRad Gene Pulser Xcell or Amaxa Nucleofector IIb devices. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

Upon delivery of the presently disclosed genetic construct or composition to the tissue, and thereupon the vector into the cells of the mammal, the transfected cells will express the Cpf1 gRNA(s) and the Cpf1 endonuclease. The genetic construct or composition may be administered to a mammal to alter gene expression or to re-engineer or alter the genome. For example, the genetic construct or composition may be administered to a mammal to correct the dystrophin gene in a mammal. The mammal may be human, non-human primate, cow, pig, sheep, goat, antelope, bison, water buffalo, bovids, deer, hedgehogs, elephants, llama, alpaca, mice, rats, or chicken, and preferably human, cow, pig, or chicken.

The genetic construct (e.g., a vector) encoding the Cpf1 gRNA(s) and the Cpf1 endonuclease can be delivered to the mammal by DNA injection (also referred to as DNA vaccination) with and without in vivo electroporation, liposome mediated, nanoparticle facilitated, and/or recombinant vectors. The recombinant vector can be delivered by any viral mode. The viral mode can be recombinant lentivirus, recombinant adenovirus, and/or recombinant adeno-associated virus.

A presently disclosed genetic construct (e.g., a vector) or a composition comprising thereof can be introduced into a cell to genetically correct a dystrophin gene (e.g., human dystrophin gene). In certain embodiments, a presently disclosed genetic construct (e.g., a vector) or a composition comprising thereof is introduced into a myoblast cell from a DMD patient. In certain embodiments, the genetic construct (e.g., a vector) or a composition comprising thereof is introduced into a fibroblast cell from a DMD patient, and the genetically corrected fibroblast cell can be treated with MyoD to induce differentiation into myoblasts, which can be implanted into subjects, such as the damaged muscles of a subject to verify that the corrected dystrophin protein is functional and/or to treat the subject. The modified cells can also be stem cells, such as induced pluripotent stem cells, bone marrow-derived progenitors, skeletal muscle progenitors, human skeletal myoblasts from DMD patients, CD 133+ cells, mesoangioblasts, and MyoD- or Pax7-transduced cells, or other myogenic progenitor cells. For example, the CRISPR/Cpf1-based gene editing system may cause neuronal or myogenic differentiation of an induced pluripotent stem cell.

### 13. Routes of Administration

The presently disclosed genetic constructs (e.g., vectors) or a composition comprising thereof may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. In certain embodiments, the presently disclosed genetic construct (e.g., a vector) or a composition is administered to a subject (e.g., a subject suffering from DMD) intramuscularly, intravenously or a combination thereof. For veterinary use, the presently disclosed genetic constructs (e.g., vectors) or compositions may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gone guns”, or other physical methods such as electroporation (“EP”), “hydrodynamic method”, or ultrasound.

The presently disclosed genetic construct (e.g., a vector) or a composition may be delivered to the mammal by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the skeletal muscle or cardiac muscle. For example, the composition may be injected into the tibialis anterior muscle or tail.

In some embodiments, the presently disclosed genetic construct (e.g., a vector) or a composition thereof is administered by 1) tail vein injections (systemic) into adult mice; 2) intramuscular injections, for example, local injection into a muscle such as the TA or gastrocnemius in adult mice; 3) intraperitoneal injections into P2 mice; or 4) facial vein injection (systemic) into P2 mice.

### 14. Cell Types

Any of these delivery methods and/or routes of administration can be utilized with a myriad of cell types, for example, those cell types currently under investigation for cell-based therapies of DMD, including, but not limited to, immortalized myoblast cells, such as wild-type and DMD patient derived lines, for example A48-50 DMD, DMD 6594 (del48-50), DMD 8036 (del48-50), C25C14 and DMD-7796 cell lines, primal DMD dermal fibroblasts, induced pluripotent stem cells, bone marrow-derived progenitors, skeletal muscle progenitors, human skeletal myoblasts from DMD patients, CD 133⁺ cells, mesoangioblasts, cardiomyocytes, hepatocytes, chondrocytes, mesenchymal progenitor cells, hematopoetic stem cells, smooth muscle cells, and MyoD- or Pax7-transduced cells, or other myogenic progenitor cells. Immortalization of human myogenic cells can be used for clonal derivation of genetically corrected myogenic cells. Cells can be modified ex vivo to isolate and expand clonal populations of immortalized DMD myoblasts that include a genetically corrected dystrophin gene and are free of other nuclease-introduced mutations in protein coding regions of the genome. Alternatively, transient in vivo delivery of CRISPR/Cpf1-based systems by non-viral or non-integrating viral gene transfer, or by direct delivery of purified proteins and gRNAs containing cell-penetrating motifs may enable highly specific correction in situ with minimal or no risk of exogenous DNA integration.

### 15. Kits

Provided herein is a kit, which may be used to correct a mutated dystrophin gene. The kit comprises at least one Cpf1 gRNA for correcting a mutated dystrophin gene and instructions for using the CRISPR/Cpf1-based gene editing system. Also provided herein is a kit, which may be used for genome editing of a dystrophin gene in skeletal muscle or cardiac muscle. The kit comprises genetic constructs (e.g., vectors) or a composition comprising thereof for genome editing in skeletal muscle or cardiac muscle, as described above, and instructions for using said composition.

Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs (e.g., vectors) or a composition comprising thereof for correcting a mutated dystrophin or genome editing of a dystrophin gene in skeletal muscle or cardiac muscle may include a modified AAV vector that includes a Cpf1 gRNA(s) and a Cpf1 endonuclease, as described above, that specifically binds and cleaves a region of the dystrophin gene. The CRISPR/Cpf1-based gene editing system, as described above, may be included in the kit to specifically bind and target a particular region in the mutated dystrophin gene. The kit may further include donor DNA, a different gRNA, or a transgene, as described above.

The kit can also optionally include one or more components, such as reagents required to use the disclosed compositions or to facilitate quality control evaluations, such as standards, buffers, diluents, salts, enzymes, enzyme co-factors, substrates, detection reagents, and the like. Other components, such as buffers and solutions for the isolation and/or treatment of the cells, also can be included in the kit. The kit can additionally include one or more other controls. One or more of the components of the kit can be lyophilized, in which case the kit can further comprise reagents suitable for the reconstitution of the lyophilized components.

### 16. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present invention has multiple aspects, illustrated by the following non-limiting examples.

### Example 1

### Guide RNA Design and Material Preparation

Cpf1 from Acidaminococcus was obtained from the Addgene non-profit plasmid repository (pY010 (pcDNA3.1-hAsCpf1; “the AsCPF1 plasmid”) from Feng Zhang (Addgene plasmid #69982)). The AsCPF1 plasmid was transformed into chemically competent E. coli and amplified, after which the sequence was verified. Cpf1 guide RNAs (also known as Cpf1 crRNAs) were designed with the University of California Santa Cruz Genome Browser program to target splice sites on prevalent exon mutations in dystrophin and the BCL11a enhancer, ordered as oligomers from Integrated DNA Technologies (IDT), prepared with PCR, and column purified as previously described (Zetsche et al., Cell 163 (3): 759-71 (2015)).

Guide RNA validation. Transfections were performed with Lipofectamine 2000 in 24-well plates of HEK293 cells (ATCC) following manufacturer's recommendations. Each well received 400 ng of AsCPF1 plasmid and 100 ng U6::sgRNA PCR products. After 72 hours, cells were isolated and genomic DNA was purified with a DNeasy column (QIAGEN). Surveyor nuclease digestion (IDT) and deletion PCR was performed with primers flanking the genomic region of interest as previously described (Ousterout et al., Nature Communications 6:6244 (2015); Guschin et al., Methods Mol. Biol. 649:247-256 (2010)). Digested PCR products were electrophoresed in TBE gels (Invitrogen) for 30 min at 200V, stained with ethidium bromide (EtBr), and imaged on a Gel Doc™ (Biorad). Deletion PCR products were electrophoresed in 1% agarose gels for 30 min at 120V, stained with EtBr, and imaged on a Gel Doc™ (Biorad).

### Example 2

### Dystrophin Splice-Acceptor Guide RNA

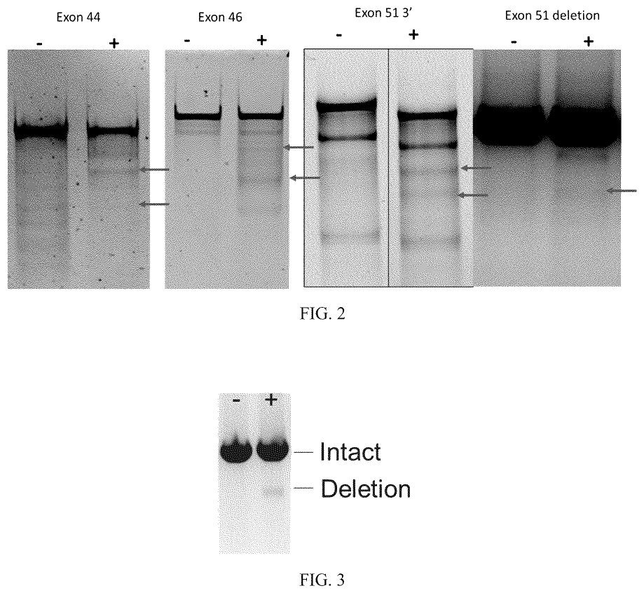

15 guide RNAs targeting the top-ranking highly-mutated dystrophin exons were designed by targeting the cut region as close to the splice acceptor as possible permitted by the presence of an available PAM (Table 1). If possible, multiple guide RNAs were targeted to the same splice acceptor. Candidate guide RNAs were screened in vitro. Guide RNAs that showed immediate positive results include those targeting exon 44, exon 46, and exon 51 (FIGS. 2A-2C). Surveyor nuclease digestion was detected in guide RNAs targeting exon 44 splice acceptor (FIG. 2A), exon 46 splice acceptor (FIG. 2B), and the 3′ end of exon 51 (FIG. 2C). FIG. 2D shows that genetic deletions can be created with a guide RNA targeting the splice acceptor of exon 51 and the 3′ end of exon 51 implying activity of the exon 51 targeted guide RNA.

Table 1 shows the design of guide RNAs targeting dystrophin exons. The PAM sequence (TTTN) is underlined. Sense guide-RNAs have TTTN on 5′ end. Guide RNAs on the antisense strand have NAAA PAMs on the 3′ end.

TABLE 1

| | | SEQ | |
|---|---|---|---|
| | | ID | Target |
| Target Sequence | Guide RNA | NO: | Exon |
| TTTGCAAAAACCCAAAATATTTTAG | CAAAAACCCAAAATATTTTAG | 36 | Exon |
| CT (SEQ ID NO: 1) | CT | | 51 |
| TTTGCCTTTTTGGTATCTTACAGGA | CCTTTTTGGTATCTTACAGGA | 37 | Exon |
| AC (SEQ ID NO: 2) | AC | | 45 |
| TCCAGGATGGCATTGGGCAGCGGC | CCGCTGCCCAATGCCATCCTG | 38 | Exon |
| AAA (SEQ ID NO: 3) | GA | | 45 |
| TTTATTTTTCCTTTTATTCTAGTTGA | TTTTTCCTTTTATTCTAGTTGA | 39 | Exon |
| A (SEQ ID NO: 4) | A | | 53 |
| TTTCTTGATCCATATGCTTTTACCTG | TTGATCCATATGCTTTTACCT | 40 | Exon |
| C (SEQ ID NO: 5 ) | GC | | 44 |
| AGGCGATTTGACAGATCTGTTGAGA | TCAACAGATCTGTCAAATCGC | 41 | Exon |
| AA (SEQ ID NO: 6) | CT | | 44 |
| TTTATTCTTCTTTCTCCAGGCTAGAA | TTCTTCTTTCTCCAGGCTAGA | 42 | Exon |
| G (SEQ ID NO: 7) | AG | | 46 |
| TTCTTTCTCCAGGCTAGAAGAACAA | GTTCTTCTAGCCTGGAGAAAG | 43 | Exon |
| AA (SEQ ID NO: 8) | AA | | 46 |
| TACAGGCAACAATGCAGGATTTGG | CAAATCCTGCATTGTTGCCTG | 44 | Exon |
| AAC (SEQ ID NO: 9) | TA | | 52 |
| TTTTCTGTTAAAGAGGAAGTTAGAA | CTGTTAAAGAGGAAGTTAGA | 45 | Exon |
| GA (SEQ ID NO: 10) | AGA | | 50 |
| TTTTAAAATTTTTATATTACAGAAT | AAAATTTTTATATTACAGAAT | 46 | Exon |
| AT (SEQ ID NO: 11) | AT | | 43 |
| AGAATATAAAAGATAGTCTACAA | TTGTAGACTATCTTTTATATTC | 47 | Exon |
| CAAA (SEQ ID NO: 12) | T | | 43 |
| TTTATTTTGCATTTTAGATGAAAGA | TTTTGCATTTTAGATGAAAGA | 48 | Exon 2 |
| GA (SEQ ID NO: 13) | GA | | |
| TTTTAGATGAAAGAGAAGATGTTCA | AACATCTTCTCTTTCATCTAA | 49 | Exon 2 |
| AA (SEQ ID NO: 14) | AA | | |
| GATGAAAGAGAAGATGTTCAAAAG | TTTTGAACATCTTCTCTTTCAT | 50 | Exon 2 |
| AAA (SEQ ID NO: 15) | C | | |

### Example 3

### Matched Overhang Deletions of Exon 51

To determine if guide RNAs with matched overhang sequences encourage seamless deletions, 6 guide RNAs were designed within intron 50 and 7 guide RNAs were designed within intron 51 (Table 2) to generate matched overhang deletions. 42 unique gRNA pairs (6x7) were tested and screened for deletion activity, i.e., targeting exon 51 deletions. Included within this set were three overhang-matched pairs (see Table 2). 7 pairs were validated for activity. FIG. 3 is a representative image showing a smaller band indicating the deletion of exon 51. These results show for the first time Cpf1-targeted splice-acceptor disruption and deletion of exon 51 of the dystrophin gene.

TABLE 2
CPF1 guide RNAs targeting regions flanking exon 51

| | | SEQ | | |
|---|---|---|---|---|
| | | ID | | |
| TARGET SEQUENCE | Guide RNA | NO: | TARGET | |
| TTTGCAAAAACCCAAAATATTTTAGCT | CAAAAACCCAAAATATTTTAGCT | 51 | Intron 50 | |
| (SEQ ID NO: 16) | | | | |
| TTTAGCTTGTGTTTCTAATTTTTCTTT | GCTTGTGTTTCTAATTTTTCTTT | 52 | Intron 50 | |
| (SEQ ID NO: 17) | | | | |
| TTTGACTTATTGTTATTGAAATTGGCT | ACTTATTGTTATTGAAATTGGCT | 53 | Intron 50 | |
| (SEQ ID NO: 18) | | | | |
| TTTCTACCATGTATTGCTAAACAAAGT | TACCATGTATTGCTAAACAAAGT | 54 | Intron 50 | Matched |
| (SEQ ID NO: 19) | | | | pair 1 |
| TTTAGTATCAATTCACACCAGCAAGTT | GTATCAATTCACACCAGCAAGTT | 55 | Intron 50 | Matched |
| (SEQ ID NO: 20) | | | | pair 2 |
| ATAATCGCCACTTTACAGAGGAGTAAA | CTCCTCTGTAAAGTGGCGATTAT | 56 | Intron 50 | Matched |
| (SEQ ID NO: 21) | | | | pair 3 |
| TTTCTTTAAAATGAAGATTTTCCACCA | TTTAAAATGAAGATTTTCCACCA | 57 | Intron 51 | |
| (SEQ ID NO: 22) | | | | |
| TTTAAAATGAAGATTTTCCACCAATCA | AAATGAAGATTTTCCACCAATCA | 58 | Intron 51 | |
| (SEQ ID NO: 23) | | | | |
| TTTTCCACCAATCACTTTACTCTCCTA | CCACCAATCACTTTACTCTCCTA | 59 | Intron 51 | |
| (SEQ ID NO: 24) | | | | |
| TTTCCCACCAGTTCTTAGGCAACTGTT | CCACCAGTTCTTAGGCAACTGTT | 60 | Intron 51 | |
| (SEQ ID NO: 25) | | | | |
| ATAATCAAGGATATAAATTAATGCAAA | CATTAATTTATATCCTTGATTAT | 61 | Intron 51 | Matched |
| (SEQ ID NO: 26) | | | | pair 3 |
| TTTTGTTGTTGTTGTTAAGGTCAAAGT | GTTGTTGTTGTTAAGGTCAAAGT | 62 | Intron 51 | Matched |
| (SEQ ID NO: 27) | | | | pair 1 |
| TTTAAAATTACCCTAGATCTTAAAGTT | AAATTACCCTAGATCTTAAAGTT | 63 | Intron 51 | Matched |
| (SEQ ID NO: 28) | | | | pair 2 |

### Example 4

### Targeted Deletion of Exon 51 in Patient Derived Myoblasts

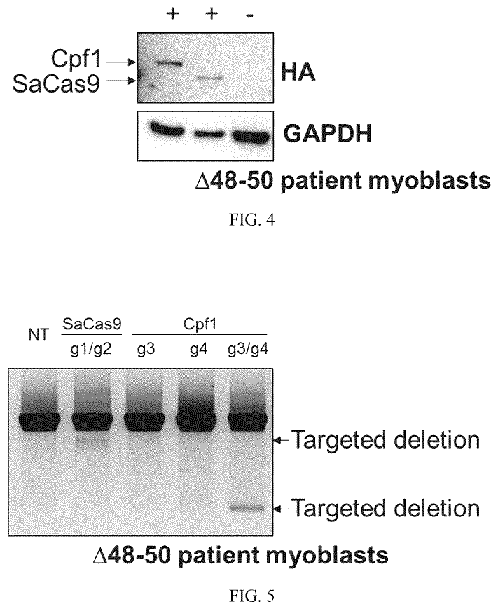

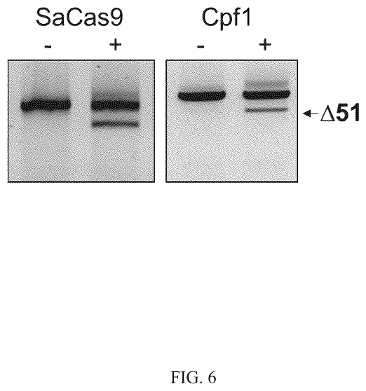

Patient derived myoblasts with an exon 48-50 deletion (Δ48-50) were cultured in skeletal muscle growth media. Electroporations were conducted according to standard lab procedure. Cells were cultured for 3 days and evaluated for protein expression (FIG. 4) and genomic deletion generated by SaCas9 (Cas9 from Staphylococcus aureus) or LbCpf1 (CPF1 from Lachnospiraceae bacterium ND2006) in patient myoblasts (FIG. 5). FIG. 4 shows a western blot for the HA-tagged SaCas9 and LbCpf1 show expression in extracted protein 72 hours after plasmid transfection. FIG. 5 shows PCR across the targeted genomic region shows a smaller band in bulk-treated myoblasts with SaCas9 gRNAs or Cpf1 crRNAs consistent with removal of exon 51 and portions of the surrounding introns.

Myoblasts were then differentiated and evaluated for dystrophin transcript expression and deletion of exon 51 (FIG. 6). FIG. 6 shows that differentiated myoblasts expressed a dystrophin transcript with an absent exon 51 as indicated by the smaller bands produced by RT-PCR, thus indicating that SaCas9 or LbCpf1 targeting of exon 51 removed exon 51 exon from the transcript.

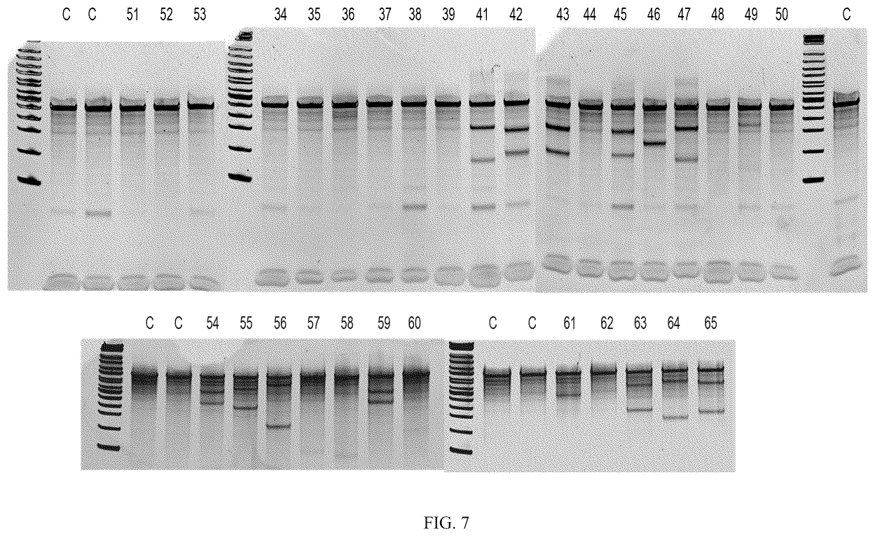

A large panel of Cpf1 crRNAs were evaluated in HEK293 cells (FIG. 7; see Table 3 for Cpf1 crRNA sequences). All of the Cpf1 crRNAs targeting exon 51 or surrounding introns used are listed in Table 3. As shown in FIG. 7, HEK293 cells treated for 3 days with a panel of crRNAs showed variable activity by the Surveyor® nuclease assay. Cpf1 crRNAs #38, 41, 42, 43, 45, 46, 47, 49, 54, 55, 56, 59, 63, 64, and 65 showed the highest activity indicated by shorted bands.

TABLE 3
crRNA sequences.

| # | crRNA Sequence | SEQ ID NO: |
|---|---|---|
| 12 | TTCCATTCTAATGGGTGGCT | 71 |
| | GTT | |
| 13 | CTCCTCTGTAAAGTGGCGAT | 72 |
| 14 | TTCCATTCTAATGGGTGGCT | 73 |
| 15 | GTATCAATTCACACCAGCAA | 74 |
| 16 | TACCATGTATTGCTAAACAA | 75 |
| 17 | ACTTATTGTTATTGAAATTG | 76 |
| 18 | GCTTGTGTTTCTAATTTTTC | 77 |
| 19 | CAAAAACCCAAAATATTTTA | 78 |
| 20 | TTTAAAATGAAGATTTTCCA | 79 |
| 21 | AAATGAAGATTTTCCACCAA | 80 |
| 22 | CCACCAATCACTTTACTCTC | 81 |
| 23 | CCACCAGTTCTTAGGCAACT | 82 |
| 24 | CATTAATTTATATCCTTGAT | 83 |
| 25 | AGTTATAGCTCTCTTTCAAT | 84 |
| 26 | ATGTATAACAATTCCAACAT | 85 |
| 27 | AAATTACCCTAGATCTTAAA | 86 |
| 28 | GTTGTTGTTGTTAAGGTCAA | 87 |
| 34 | GCTTGTGTTTCTAATTTTTC | 88 |
| 35 | TAATTTTTCTTTTTCTTCTT | 89 |
| 36 | GCAAAAAGGAAAAAAGAAGA | 90 |
| 37 | GGGTTTTTGCAAAAAGGAAA | 91 |
| 38 | AGCTCCTACTCAGACTGTTA | 92 |
| 39 | TGCAAAAACCCAAAATATTT | 93 |
| 40 | TGTCACCAGAGTAACAGTCT | 94 |
| 41 | CTTAGTAACCACAGGTTGTG | 95 |
| 42 | TAGTTTGGAGATGGCAGTTT | 96 |
| 43 | GAGATGGCAGTTTCCTTAGT | 97 |
| 44 | CTTGATGTTGGAGGTACCTG | 98 |
| 45 | ATGTTGGAGGTACCTGCTCT | 99 |
| 46 | TAACTTGATCAAGCAGAGAA | 100 |
| 47 | TCTGCTTGATCAAGTTATAA | 101 |
| 48 | TAAAATCACAGAGGGTGATG | 102 |
| 49 | ATATCCTCAAGGTCACCCAC | 103 |
| 50 | ATGATCATCTCGTTGATATC | 104 |
| 51 | TCATACCTTCTGCTTGATGA | 105 |
| 52 | TCATTTTTTCTCATACCTTC | 106 |
| 53 | TGCCAACTTTTATCATTTTT | 107 |
| 54 | AATCAGAAAGAAGATCTTAT | 108 |
| 55 | ATTTCCCTAGGGTCCAGCTT | 109 |
| 56 | GCTCAAATTGTTACTCTTCA | 110 |
| 57 | AGCTCCTACTCAGACTGTTA | 111 |
| 58 | ATTCTAGTACTATGCATCTT | 112 |
| 59 | ACTTAAGTTACTTGTCCAGG | 113 |
| 60 | CCAAGGTCCCAGAGTTCCTA | 114 |
| 61 | TTTCCCTGGCAAGGTCTGAA | 115 |
| 62 | GCTCATTCTCATGCCTGGAC | 116 |
| 63 | TTTAGCAATACATGGTAGAA | 117 |
| 64 | AGCCAAACTCTTATTCATGA | 118 |
| 65 | TAACAATGTGGATACTTTGT | 119 |

### Example 5

### BCL11a Enhancer Targeting

Potential candidate for increasing fetal globin levels in sickle cell anemia (SCA) were designed. Guide RNAs for Cpf1 were designed to target the BCL11a enhancer region (Table 3) in order to generate potential candidate for increasing fetal globin levels in sickle cell anemia (SCA). These reagents were designed to disrupt the BCL11a enhancer. These reagents will be tested in cell models of SCA.

TABLE 4
guide RNAs targeting the human
BCL1la enhancer region

| | | SEQ |
|---|---|---|
| | | ID |
| Target Sequence | Guide RNA | NO |
| CACGCCCCCACCCTAATCAGAGGCCAA | GCCTCTGATTAG | 64 |
| A (SEQ ID NO: 29) | GGTGGGGGCGTG | |
| CCAAACCCTTCCTGGAGCCTGTGATAA | TCACAGGCTCCA | 65 |
| A (SEQ ID NO: 30) | GGAAGGGTTTGG | |
| CCTTCCGAAAGAGGCCCCCCTGGGCAA | CCCAGGGGGGCC | 66 |
| A (SEQ ID NO: 31) | TCTTTCGGAAGG | |
| TCTCCATCACCAAGAGAGCCTTCCGAA | GGAAGGCTCTCT | 67 |
| A (SEQ ID NO: 32) | TGGTGATGGAGA | |
| TGTTAGCTTGCACTAGACTAGCTTCAA | AAGCTAGTCTAG | 68 |
| A (SEQ ID NO: 33) | TGCAAGCTAACA | |
| TTTTCTGGCCTATGTTATTACCTGTAT | CTGGCCTATGTT | 69 |
| G (SEQ ID NO: 34) | ATTACCTGTATG | |
| TTTCTGGCCTATGTTATTACCTGTATG | TGGCCTATGTTA | 70 |
| G (SEQ ID NO: 35) | TTACCTGTATGG | |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clause:

Clause 1. A Cpf1 guide RNA (gRNA) that targets a dystrophin gene and comprises a polynucleotide sequence corresponding to at least one of SEQ ID NOs: 36-64, 71-119, or a complement thereof.

Clause 2. A DNA targeting composition comprising a Cpf1 endonuclease and at least one Cpf1 gRNA of clause 1.

Clause 3. A DNA targeting composition comprising a first Cpf1 gRNA and a second Cpf1 gRNA, the first Cpf1 gRNA and the second Cpf1 gRNA each comprising a polynucleotide sequence corresponding to at least one of SEQ ID NOs: 36-64, 71-119, or a complement thereof, wherein the first Cpf1 gRNA and the second Cpf1 gRNA comprise different polynucleotide sequences, and wherein the first Cpf1 gRNA and the second Cpf1 gRNA target a dystrophin gene.

Clause 4. The DNA targeting composition of clause 3, wherein the first Cpf1 gRNA comprises a polynucleotide sequence corresponding to SEQ ID NO: 54, SEQ ID NO: 55, or SEQ ID NO: 56, and the second Cpf1 gRNA comprises a polynucleotide sequence corresponding to SEQ ID NO: 62, SEQ ID NO: 63, or SEQ ID NO: 61.

Clause 5. The DNA targeting composition of clause 3 or 4, wherein the first Cpf1 gRNA and the second Cpf1 gRNA are selected from the group consisting of: (i) a first Cpf1 gRNA comprising a polynucleotide sequence set forth in SEQ ID NO: 54 and a second Cpf1 gRNA comprising a polynucleotide sequence set forth in SEQ ID NO: 62; (ii) a first Cpf1 gRNA comprising a polynucleotide sequence set forth in SEQ ID NO: 55 and a second Cpf1 gRNA comprising a polynucleotide sequence set forth in SEQ ID NO: 63; and (iii) a first Cpf1 gRNA comprising a polynucleotide sequence set forth in SEQ ID NO: 56 and a second Cpf1 gRNA comprising a polynucleotide sequence set forth in SEQ ID NO: 61.

Clause 6. The DNA targeting composition of any one of clauses 3 to 5, further comprising a Cpf1 endonuclease.

Clause 7. The DNA targeting composition of clause 2 or 6, wherein the Cpf1 endonuclease recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123).

Clause 8. The DNA targeting composition of clause 7, wherein the Cpf1 endonuclease is derived from a bacterial species selected from the group consisting of Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae.

Clause 9. The DNA targeting composition of any one of clauses 6 to 8, wherein the Cpf1 endonuclease is derived from Lachnospiraceae bacterium ND2006 (LbCpf1) or from Acidaminococcus (AsCpf1).

Clause 10. The DNA targeting composition of any one of clauses 6 to 9, wherein the Cpf1 endonuclease is encoded by a polynucleotide sequence comprising SEQ ID NO: 124 or SEQ ID NO: 125.

Clause 11. An isolated polynucleotide comprising the Cpf1 gRNA of clause 1 or a polynucleotide sequence encoding the DNA targeting composition of any one of clauses 2 to 10.

Clause 12. A vector comprising the Cpf1 gRNA of clause 1, a polynucleotide sequence encoding the DNA targeting composition of any one of clauses 2 to 10, or the isolated polynucleotide of clause 10.

Clause 13. The vector of clause 12, further comprising a polynucleotide sequence encoding a Cpf1 endonuclease.

Clause 14. A vector encoding: (a) a first Cpf1 guide RNA (gRNA), (b) a second Cpf1 gRNA, and (c) at least one Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), wherein the first Cpf1 gRNA and the second Cpf1 gRNA comprises a polynucleotide sequence corresponding to at least one of SEQ ID NOs: 36-64, 71-119, or a complement thereof, and wherein the first Cpf1 gRNA and the second Cpf1 gRNA comprise different polynucleotide sequences.

Clause 15. The vector of clause 14, wherein the vector is configured to form a first and a second double strand break in a first and a second intron flanking exon 51 of the human DMD gene.

Clause 16. The vector of clause 14 or 15, wherein the first Cpf1 gRNA and the second Cpf1 gRNA are selected from the group consisting of: (i) a first Cpf1 gRNA comprising a polynucleotide sequence set forth in SEQ ID NO: 54 and a second Cpf1 gRNA comprising a polynucleotide sequence set forth in SEQ ID NO: 62; (ii) a first Cpf1 gRNA comprising a polynucleotide sequence set forth in SEQ ID NO: 55 and a second Cpf1 gRNA comprising a polynucleotide sequence set forth in SEQ ID NO: 63; and (iii) a first Cpf1 gRNA comprising a polynucleotide sequence set forth in SEQ ID NO: 56 and a second Cpf1 gRNA comprising a polynucleotide sequence set forth in SEQ ID NO: 61.

Clause 17. The vector of any one of clauses 12 to 16, wherein the vector is a viral vector.

Clause 18. The vector of clause 17, wherein the vector is an Adeno-associated virus (AAV) vector.

Clause 19. The vector of any one of clauses 12 to 18, wherein the vector comprises a tissue-specific promoter operably linked to the polynucleotide sequence encoding the first Cpf1 gRNA, the second Cpf1 gRNA, and/or the Cpf1 endonuclease.

Clause 20. The vector of clause 19, wherein the tissue-specific promoter is a muscle specific promoter.

Clause 21. A cell comprising the Cpf1 gRNA of clause 1, a polynucleotide sequence encoding the DNA targeting composition of any one of clauses 2 to 10, the isolated polynucleotide of clause 11, or the vector of any one of clauses 12 to 20.

Clause 22. A kit comprising the Cpf1 gRNA of clause 1, a polynucleotide sequence encoding the DNA targeting composition of any one of clauses 2 to 10, the isolated polynucleotide of clause 11, the vector of any one of clauses 12 to 20, or the cell of clause 21.

Clause 23. A composition for deleting a segment of a dystrophin gene comprising exon 51, the composition comprising: (a) a first vector comprising a polynucleotide sequence encoding a first Cpf1 guide RNA (gRNA) and a polynucleotide sequence encoding a first Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), and (b) a second vector comprising a polynucleotide sequence encoding a second Cpf1 gRNA and a polynucleotide sequence encoding a second Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), wherein the first Cpf1 gRNA and the second Cpf1 gRNA comprise a polynucleotide sequence corresponding to at least one of SEQ ID NOs: 36-64, 71-119, or a complement thereof, wherein the first Cpf1 gRNA and the second Cpf1 gRNA comprise different polynucleotide sequences, and wherein the first vector and second vector are configured to form a first and a second double strand break in a first intron and a second intron flanking exon 51 of the human DMD gene, respectively, thereby deleting a segment of the dystrophin gene comprising exon 51.

Clause 24. The composition of clause 23, wherein the first Cpf1 gRNA and the second Cpf1 gRNA are selected from the group consisting of: (i) a first Cpf1 gRNA comprising a polynucleotide sequence set forth in SEQ ID NO: 54 and a second Cpf1 gRNA comprising a polynucleotide sequence set forth in SEQ ID NO: 62; (ii) a first Cpf1 gRNA comprising a polynucleotide sequence set forth in SEQ ID NO: 55 and a second Cpf1 gRNA comprising a polynucleotide sequence set forth in SEQ ID NO: 63; and (iii) a first Cpf1 gRNA comprising a polynucleotide sequence set forth in SEQ ID NO: 56 and a second Cpf1 gRNA comprising a polynucleotide sequence set forth in SEQ ID NO: 61.

Clause 25. The composition of clause 23 or 24, wherein the first Cpf1 endonuclease and the second Cpf1 endonuclease are the same.

Clause 26. The composition of clause 23 or 24, wherein the first Cpf1 endonuclease and the second Cpf1 endonuclease are different.

Clause 27. The composition of clause 25 or 26, wherein the first Cpf1 endonuclease and/or the second Cpf1 endonuclease are CPF1 endonuclease from Lachnospiraceae bacterium ND2006 (LbCpf1) and/or from Acidaminococcus (AsCpf1).

Clause 28. The composition of any one of clauses 25 to 27, wherein the first Cpf1 endonuclease and/or the second Cpf1 endonuclease are encoded by a polynucleotide sequence comprising SEQ ID NO: 124 or SEQ ID NO: 125.

Clause 29. The composition of any one of clauses 23 to 28, wherein the first vector and/or the second vector is a viral vector.

Clause 30. The composition of clause 29, wherein the first vector and/or the second vector is an Adeno-associated virus (AAV) vector.

Clause 31. The composition of clause 30, wherein the AAV vector is an AAV8 vector or an AAV9 vector.

Clause 32. The composition of any one of clauses 23 to 31, wherein the dystrophin gene is a human dystrophin gene.

Clause 33. The composition of any one of clauses 23 to 32, for use in a medicament.

Clause 34. The composition of any one of clauses 23 to 32, for use in the treatment of Duchenne Muscular Dystrophy.

Clause 35. A cell comprising the composition of any one of clauses 23 to 34.

Clause 36. A modified adeno-associated viral vector for genome editing a mutant dystrophin gene in a subject comprising a first polynucleotide sequence encoding the Cpf1 gRNA of clause 1, and a second polynucleotide sequence encoding a Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123).

Clause 37. A method of correcting a mutant dystrophin gene in a cell, the method comprising administering to a cell the Cpf1 gRNA of clause 1, a polynucleotide sequence encoding the DNA targeting composition of any one of clauses 2 to 10, the isolated polynucleotide of clause 11, the vector of any one of clauses 12 to 20, the composition of any one of clauses 23 to 34, or the modified adeno-associated viral vector of clause 36.

Clause 38. The method of clause 37, wherein correcting the mutant dystrophin gene comprises nuclease-mediated non-homologous end joining or homology-directed repair.

Clause 39. A method of genome editing a mutant dystrophin gene in a subject, the method comprising administering to the subject a genome editing composition comprising the Cpf1 gRNA of clause 1, a polynucleotide sequence encoding the DNA targeting composition of any one of clauses 2 to 10, the isolated polynucleotide of clause 11, the vector of any one of clauses 12 to 20, the composition of any one of clauses 23 to 34, or the modified adeno-associated viral vector of clause 36.

Clause 40. The method of clause 39, wherein the genome editing composition is administered to the subject intramuscularly, intravenously, or a combination thereof.

Clause 41. The method of clause 39 or 40, wherein the genome editing comprises nuclease-mediated non-homologous end joining or homology-directed repair.

Clause 42. A method of treating a subject in need thereof having a mutant dystrophin gene, the method comprising administering to the subject the Cpf1 gRNA of clause 1, a polynucleotide sequence encoding the DNA targeting composition of any one of clauses 2 to 10, the isolated polynucleotide of clause 11, the vector of any one of clauses 12 to 20, the composition of any one of clauses 23 to 34, or the modified adeno-associated viral vector of clause 36.

Clause 43. A method of correcting a mutant dystrophin gene in a cell, comprising administering to the cell: (a) a first vector comprising a polynucleotide sequence encoding a first Cpf1 guide RNA (gRNA) and a polynucleotide sequence encoding a first Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), and (b) a second vector comprising a polynucleotide sequence encoding a second Cpf1 gRNA and a polynucleotide sequence encoding a second Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), wherein the first Cpf1 gRNA and the second Cpf1 gRNA comprises a polynucleotide sequence corresponding to at least one of SEQ ID NOs: 36-64, 71-119, or a complement thereof, and the vector is configured to form a first and a second double strand break in a first and a second intron flanking exon 51 of the human dystrophin gene, respectively, thereby deleting a segment of the dystrophin gene comprising exon 51 and correcting the mutant dystrophin gene in a cell.

Clause 44. The method of clause 43, wherein the first Cpf1 gRNA and the second Cpf1 gRNA are selected from the group consisting of: (i) a first Cpf1 gRNA comprising a polynucleotide sequence set forth in SEQ ID NO: 54 and a second Cpf1 gRNA comprising a polynucleotide sequence set forth in SEQ ID NO: 62; (ii) a first Cpf1 gRNA comprising a polynucleotide sequence set forth in SEQ ID NO: 55 and a second Cpf1 gRNA comprising a polynucleotide sequence set forth in SEQ ID NO: 63; and (iii) a first Cpf1 gRNA comprising a polynucleotide sequence set forth in SEQ ID NO: 56 and a second Cpf1 gRNA comprising a polynucleotide sequence set forth in SEQ ID NO: 61.

Clause 45. The method of clause 43 or 44, wherein the mutant dystrophin gene comprises a premature stop codon, disrupted reading frame, an aberrant splice acceptor site, or an aberrant splice donor site.

Clause 46. The method of clause 45, wherein the mutant dystrophin gene comprises a frameshift mutation which causes a premature stop codon and a truncated gene product.

Clause 47. The method of clause 43 or 44, wherein the mutant dystrophin gene comprises a deletion of one or more exons which disrupts the reading frame.

Clause 48. The method of any one of clauses 43 to 47, wherein the correction of the mutant dystrophin gene comprises a deletion of a premature stop codon, correction of a disrupted reading frame, or modulation of splicing by disruption of a splice acceptor site or disruption of a splice donor sequence.

Clause 49. The method of clause 48, wherein the correction of the mutant dystrophin gene comprises deletion of exon 51.

Clause 50. The method of any one of clauses 43 to 49, wherein the correction of the mutant dystrophin gene comprises nuclease mediated non-homologous end joining or homology-directed repair.

Clause 51. The method of any one of clauses 43 to 50, wherein the cell is a myoblast cell.

Clause 52. The method of any one of clauses 43 to 51, wherein the cell is from a subject suffering from Duchenne muscular dystrophy.

Clause 53. A method of treating a subject in need thereof having a mutant dystrophin gene, the method comprising administering to the subject: (a) a first vector comprising a polynucleotide sequence encoding a first Cpf1 guide RNA (gRNA) and a polynucleotide sequence encoding a first Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), and (b) a second vector comprising a polynucleotide sequence encoding a second Cpf1 gRNA and a polynucleotide sequence encoding a second Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), wherein the first Cpf1 gRNA and the second Cpf1 gRNA comprises a polynucleotide sequence corresponding to at least one of SEQ ID NOs: 36-64, 71-119, or a complement thereof, and the first vector and the second vector are configured to form a first and a second double strand break in a first and a second intron flanking exon 51 of the human dystrophin gene, respectively, thereby deleting a segment of the dystrophin gene comprising exon 51 and treating the subject.

Clause 54. The method of clause 53, wherein the first Cpf1 gRNA and the second Cpf1 gRNA are selected from the group consisting of: (i) a first Cpf1 gRNA comprising a polynucleotide sequence set forth in SEQ ID NO: 54, and a second Cpf1 gRNA comprising a polynucleotide sequence set forth in SEQ ID NO: 62; (ii) a first Cpf1 gRNA comprising a polynucleotide sequence set forth in SEQ ID NO: 55, and a second Cpf1 gRNA comprising a polynucleotide sequence set forth in SEQ ID NO: 63; and (iii) a first Cpf1 gRNA comprising a polynucleotide sequence set forth in SEQ ID NO: Clause 56, and a second Cpf1 gRNA comprising a polynucleotide sequence set forth in SEQ ID NO: 61.

Clause 55. The method of clause 53 or 54, wherein the subject is suffering from Duchenne muscular dystrophy.

Clause 56. The method of any one of clauses 53 to 55, wherein the first vector and second vector are administered to the subject intramuscularly, intravenously, or a combination thereof.

Clause 57. A Cpf1 guide RNA (gRNA) that targets an enhancer of the B-cell lymphoma/leukemia 11A (BCL11a) gene and comprises a polynucleotide sequence corresponding to at least one of SEQ ID NOs: 65-70, or a complement thereof.

Clause 58. A method of disrupting an enhancer of a B-cell lymphoma/leukemia 11A gene in a cell, the method comprising administering to the cell at least one Cpf1 gRNA of clause 57 and a Cpf1 endonuclease.

What is claimed is:

1. A Cpf1 guide RNA (gRNA) that targets a dystrophin gene and comprises a polynucleotide sequence corresponding to at least one of SEQ ID NOs: 36-64, 71-119, or a complement thereof.

2. A DNA targeting composition comprising a Cpf1 endonuclease and at least one Cpf1 gRNA of claim 1.

3. A DNA targeting composition comprising a first Cpf1 gRNA and a second Cpf1 gRNA, the first Cpf1 gRNA and the second Cpf1 gRNA each comprising a polynucleotide sequence corresponding to at least one of SEQ ID NOs: 36-64, 71-119, or a complement thereof, wherein the first Cpf1 gRNA and the second Cpf1 gRNA comprise different polynucleotide sequences, and wherein the first Cpf1 gRNA and the second Cpf1 gRNA target a dystrophin gene.

4. The DNA targeting composition of claim 3, wherein the first Cpf1 gRNA comprises a polynucleotide sequence corresponding to SEQ ID NO: 54, SEQ ID NO: 55, or SEQ ID NO: 56, and the second Cpf1 gRNA comprises a polynucleotide sequence corresponding to SEQ ID NO: 62, SEQ ID NO: 63, or SEQ ID NO: 61.

5. The DNA targeting composition of claim 3 or 4, wherein the first Cpf1 gRNA and the second Cpf1 gRNA are selected from the group consisting of:
(i) a first Cpf1 gRNA comprising a polynucleotide sequence set forth in SEQ ID NO: 54 and a second Cpf1 gRNA comprising a polynucleotide sequence set forth in SEQ ID NO: 62;
(ii) a first Cpf1 gRNA comprising a polynucleotide sequence set forth in SEQ ID NO: 55 and a second Cpf1 gRNA comprising a polynucleotide sequence set forth in SEQ ID NO: 63; and
(iii) a first Cpf1 gRNA comprising a polynucleotide sequence set forth in SEQ ID NO: 56 and a second Cpf1 gRNA comprising a polynucleotide sequence set forth in SEQ ID NO: 61.

6. The DNA targeting composition of any one of claims 3 to 5, further comprising a Cpf1 endonuclease.

7. The DNA targeting composition of claim 2 or 6, wherein the Cpf1 endonuclease recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123).

8. The DNA targeting composition of claim 7, wherein the Cpf1 endonuclease is derived from a bacterial species selected from the group consisting of Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2 44 17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae.

9. The DNA targeting composition of any one of claims 6 to 8, wherein the Cpf1 endonuclease is derived from Lachnospiraceae bacterium ND2006 (LbCpf1) or from Acidaminococcus (AsCpf1).

10. The DNA targeting composition of any one of claims 6 to 9, wherein the Cpf1 endonuclease is encoded by a polynucleotide sequence comprising SEQ ID NO: 124 or SEQ ID NO: 125.

11. An isolated polynucleotide comprising the Cpf1 gRNA of claim 1 or a polynucleotide sequence encoding the DNA targeting composition of any one of claims 2 to 10.

12. A vector comprising the Cpf1 gRNA of claim 1, a polynucleotide sequence encoding the DNA targeting composition of any one of claims 2 to 10, or the isolated polynucleotide of claim 10.

13. The vector of claim 12, further comprising a polynucleotide sequence encoding a Cpf1 endonuclease.

14. A vector encoding:
(a) a first Cpf1 guide RNA (gRNA),
(b) a second Cpf1 gRNA, and
(c) at least one Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123),
wherein the first Cpf1 gRNA and the second Cpf1 gRNA comprises a polynucleotide sequence corresponding to at least one of SEQ ID NOs: 36-64, 71-119, or a complement thereof, and wherein the first Cpf1 gRNA and the second Cpf1 gRNA comprise different polynucleotide sequences.

15. The vector of claim 14, wherein the vector is configured to form a first and a second double strand break in a first and a second intron flanking exon 51 of the human DMD gene.

16. The vector of claim 14 or 15, wherein the first Cpf1 gRNA and the second Cpf1 gRNA are selected from the group consisting of:
(i) a first Cpf1 gRNA comprising a polynucleotide sequence set forth in SEQ ID NO: 54 and a second Cpf1 gRNA comprising a polynucleotide sequence set forth in SEQ ID NO: 62;
(ii) a first Cpf1 gRNA comprising a polynucleotide sequence set forth in SEQ ID NO: 55 and a second Cpf1 gRNA comprising a polynucleotide sequence set forth in SEQ ID NO: 63; and
iii) a first Cpf1 gRNA comprising a polynucleotide sequence set forth in SEQ ID NO: 56 and a second Cpf1 gRNA comprising a polynucleotide sequence set forth in SEQ ID NO: 61.

17. The vector of any one of claims 12 to 16, wherein the vector is a viral vector.

18. The vector of claim 17, wherein the vector is an Adeno-associated virus (AAV) vector.

19. The vector of any one of claims 12 to 18, wherein the vector comprises a tissue-specific promoter operably linked to the polynucleotide sequence encoding the first Cpf1 gRNA, the second Cpf1 gRNA, and/or the Cpf1 endonuclease.

20. The vector of claim 19, wherein the tissue-specific promoter is a muscle specific promoter.

21. A cell comprising the Cpf1 gRNA of claim 1, a polynucleotide sequence encoding the DNA targeting composition of any one of claims 2 to 10, the isolated polynucleotide of claim 11, or the vector of any one of claims 12 to 20.

22. A kit comprising the Cpf1 gRNA of claim 1, a polynucleotide sequence encoding the DNA targeting composition of any one of claims 2 to 10, the isolated polynucleotide of claim 11, the vector of any one of claims 12 to 20, or the cell of claim 21.

23. A composition for deleting a segment of a dystrophin gene comprising exon 51, the composition comprising:
(a) a first vector comprising a polynucleotide sequence encoding a first Cpf1 guide RNA (gRNA) and a polynucleotide sequence encoding a first Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), and
(b) a second vector comprising a polynucleotide sequence encoding a second Cpf1 gRNA and a polynucleotide sequence encoding a second Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123),
wherein the first Cpf1 gRNA and the second Cpf1 gRNA comprise a polynucleotide sequence corresponding to at least one of SEQ ID NOs: 36-64, 71-119, or a complement thereof, wherein the first Cpf1 gRNA and the second Cpf1 gRNA comprise different polynucleotide sequences, and wherein the first vector and second vector are configured to form a first and a second double strand break in a first intron and a second intron flanking exon 51 of the human DMD gene, respectively, thereby deleting a segment of the dystrophin gene comprising exon 51.

24. The composition of claim 23, wherein the first Cpf1 gRNA and the second Cpf1 gRNA are selected from the group consisting of:
(i) a first Cpf1 gRNA comprising a polynucleotide sequence set forth in SEQ ID NO: 54 and a second Cpf1 gRNA comprising a polynucleotide sequence set forth in SEQ ID NO: 62;
(ii) a first Cpf1 gRNA comprising a polynucleotide sequence set forth in SEQ ID NO: 55 and a second Cpf1 gRNA comprising a polynucleotide sequence set forth in SEQ ID NO: 63; and
(iii) a first Cpf1 gRNA comprising a polynucleotide sequence set forth in SEQ ID NO: 56 and a second Cpf1 gRNA comprising a polynucleotide sequence set forth in SEQ ID NO: 61.

25. The composition of claim 23 or 24, wherein the first Cpf1 endonuclease and the second Cpf1 endonuclease are the same.

26. The composition of claim 23 or 24, wherein the first Cpf1 endonuclease and the second Cpf1 endonuclease are different.

27. The composition of claim 25 or 26, wherein the first Cpf1 endonuclease and/or the second Cpf1 endonuclease are CPF1 endonuclease from Lachnospiracede bacterium ND2006 (LbCpf1) and/or from Acidaminococcus (AsCpf1).

28. The composition of any one of claims 25 to 27, wherein the first Cpf1 endonuclease and/or the second Cpf1 endonuclease are encoded by a polynucleotide sequence comprising SEQ ID NO: 124 or SEQ ID NO: 125.

29. The composition of any one of claims 23 to 28, wherein the first vector and/or the second vector is a viral vector.

30. The composition of claim 29, wherein the first vector and/or the second vector is an Adeno-associated virus (AAV) vector.

31. The composition of claim 30, wherein the AAV vector is an AAV8 vector or an AAV9 vector.

32. The composition of any one of claims 23 to 31, wherein the dystrophin gene is a human dystrophin gene.

33. The composition of any one of claims 23 to 32, for use in a medicament.

34. The composition of any one of claims 23 to 32, for use in the treatment of Duchenne Muscular Dystrophy.

35. A cell comprising the composition of any one of claims 23 to 34.

36. A modified adeno-associated viral vector for genome editing a mutant dystrophin gene in a subject comprising a first polynucleotide sequence encoding the Cpf1 gRNA of claim 1, and a second polynucleotide sequence encoding a Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123).

37. A method of correcting a mutant dystrophin gene in a cell, the method comprising administering to a cell the Cpf1 gRNA of claim 1, a polynucleotide sequence encoding the DNA targeting composition of any one of claims 2 to 10, the isolated polynucleotide of claim 11, the vector of any one of claims 12 to 20, the composition of any one of claims 23 to 34, or the modified adeno-associated viral vector of claim 36.

38. The method of claim 37, wherein correcting the mutant dystrophin gene comprises nuclease-mediated non-homologous end joining or homology-directed repair.

39. A method of genome editing a mutant dystrophin gene in a subject, the method comprising administering to the subject a genome editing composition comprising the Cpf1 gRNA of claim 1, a polynucleotide sequence encoding the DNA targeting composition of any one of claims 2 to 10, the isolated polynucleotide of claim 11, the vector of any one of claims 12 to 20, the composition of any one of claims 23 to 34, or the modified adeno-associated viral vector of claim 36.

40. The method of claim 39, wherein the genome editing composition is administered to the subject intramuscularly, intravenously, or a combination thereof.

41. The method of claim 39 or 40, wherein the genome editing comprises nuclease-mediated non-homologous end joining or homology-directed repair.

42. A method of treating a subject in need thereof having a mutant dystrophin gene, the method comprising administering to the subject the Cpf1 gRNA of claim 1, a polynucleotide sequence encoding the DNA targeting composition of any one of claims 2 to 10, the isolated polynucleotide of claim 11, the vector of any one of claims 12 to 20, the composition of any one of claims 23 to 34, or the modified adeno-associated viral vector of claim 36.

43. A method of correcting a mutant dystrophin gene in a cell, comprising administering to the cell:
(a) a first vector comprising a polynucleotide sequence encoding a first Cpf1 guide RNA (gRNA) and a polynucleotide sequence encoding a first Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), and
(b) a second vector comprising a polynucleotide sequence encoding a second Cpf1 gRNA and a polynucleotide sequence encoding a second Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123),
wherein the first Cpf1 gRNA and the second Cpf1 gRNA comprises a polynucleotide sequence corresponding to at least one of SEQ ID NOs: 36-64, 71-119, or a complement thereof, and the vector is configured to form a first and a second double strand break in a first and a second intron flanking exon 51 of the human dystrophin gene, respectively, thereby deleting a segment of the dystrophin gene comprising exon 51 and correcting the mutant dystrophin gene in a cell.

44. The method of claim 43, wherein the first Cpf1 gRNA and the second Cpf1 gRNA are selected from the group consisting of:
(i) a first Cpf1 gRNA comprising a polynucleotide sequence set forth in SEQ ID NO: 54 and a second Cpf1 gRNA comprising a polynucleotide sequence set forth in SEQ ID NO: 62;
(ii) a first Cpf1 gRNA comprising a polynucleotide sequence set forth in SEQ ID NO: 55 and a second Cpf1 gRNA comprising a polynucleotide sequence set forth in SEQ ID NO: 63; and
(iii) a first Cpf1 gRNA comprising a polynucleotide sequence set forth in SEQ ID NO: 56 and a second Cpf1 gRNA comprising a polynucleotide sequence set forth in SEQ ID NO: 61.

45. The method of claim 43 or 44, wherein the mutant dystrophin gene comprises a premature stop codon, disrupted reading frame, an aberrant splice acceptor site, or an aberrant splice donor site.

46. The method of claim 45, wherein the mutant dystrophin gene comprises a frameshift mutation which causes a premature stop codon and a truncated gene product.

47. The method of claim 43 or 44, wherein the mutant dystrophin gene comprises a deletion of one or more exons which disrupts the reading frame.

48. The method of any one of claims 43 to 47, wherein the correction of the mutant dystrophin gene comprises a deletion of a premature stop codon, correction of a disrupted reading frame, or modulation of splicing by disruption of a splice acceptor site or disruption of a splice donor sequence.

49. The method of claim 48, wherein the correction of the mutant dystrophin gene comprises deletion of exon 51.

50. The method of any one of claims 43 to 49, wherein the correction of the mutant dystrophin gene comprises nuclease mediated non-homologous end joining or homology-directed repair.

51. The method of any one of claims 43 to 50, wherein the cell is a myoblast cell.

52. The method of any one of claims 43 to 51, wherein the cell is from a subject suffering from Duchenne muscular dystrophy.

53. A method of treating a subject in need thereof having a mutant dystrophin gene, the method comprising administering to the subject:
(a) a first vector comprising a polynucleotide sequence encoding a first Cpf1 guide RNA (gRNA) and a polynucleotide sequence encoding a first Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123), and
(b) a second vector comprising a polynucleotide sequence encoding a second Cpf1 gRNA and a polynucleotide sequence encoding a second Cpf1 endonuclease that recognizes a Protospacer Adjacent Motif (PAM) of TTTA (SEQ ID NO: 120), TTTG (SEQ ID NO: 121), TTTC (SEQ ID NO: 122), or TTTT (SEQ ID NO: 123),
wherein the first Cpf1 gRNA and the second Cpf1 gRNA comprises a polynucleotide sequence corresponding to at least one of SEQ ID NOs: 36-64, 71-119, or a complement thereof, and the first vector and the second vector are configured to form a first and a second double strand break in a first and a second intron flanking exon 51 of the human dystrophin gene, respectively, thereby deleting a segment of the dystrophin gene comprising exon 51 and treating the subject.

54. The method of claim 53, wherein the first Cpf1 gRNA and the second Cpf1 gRNA are selected from the group consisting of:
(i) a first Cpf1 gRNA comprising a polynucleotide sequence set forth in SEQ ID NO: 54, and a second Cpf1 gRNA comprising a polynucleotide sequence set forth in SEQ ID NO: 62;
(ii) a first Cpf1 gRNA comprising a polynucleotide sequence set forth in SEQ ID NO: 55, and a second Cpf1 gRNA comprising a polynucleotide sequence set forth in SEQ ID NO: 63; and
(iii) a first Cpf1 gRNA comprising a polynucleotide sequence set forth in SEQ ID NO: 56, and a second Cpf1 gRNA comprising a polynucleotide sequence set forth in SEQ ID NO: 61.

55. The method of claim 53 or 54, wherein the subject is suffering from Duchenne muscular dystrophy.

56. The method of any one of claims 53 to 55, wherein the first vector and second vector are administered to the subject intramuscularly, intravenously, or a combination thereof.

57. A Cpf1 guide RNA (gRNA) that targets an enhancer of the B-cell lymphoma/leukemia 11A (BCL11a) gene and comprises a polynucleotide sequence corresponding to at least one of SEQ ID NOs: 65-70, or a complement thereof.

58. A method of disrupting an enhancer of a B-cell lymphoma/leukemia 11A gene in a cell, the method comprising administering to the cell at least one Cpf1 gRNA of claim 57 and a Cpf1 endonuclease.
